(19) United States
(12) Patent Application Publication
(10) Pub. No.: US 2024/0315822 A1
(43) Pub. Date: Sep. 26, 2024

(54) ESTROUS SENSOR IMPLANTABLE INTO BODY OF DOMESTIC ANIMAL AND ESTRUS DETERMINATION METHOD USING THE SAME

(71) Applicant: DAMOATECH CO.,LTD., Seongnam-si (KR)

(72) Inventor: Hu Min JUNG, Hanam-si (KR)

(21) Appl. No.: 18/677,107

(22) Filed: May 29, 2024

Related U.S. Application Data
(63) Continuation of application No. PCT/KR2021/017703, filed on Nov. 29, 2021.

(51) Int. Cl. A61D 17/00; A61D 13/00; A61D 99/00

(52) U.S. Cl. CPC A61D 17/002; A61D 13/00; A61D 99/00

(57) ABSTRACT

An estrous sensor, which is implanted into a body of a domestic animal and detects estrus of the domestic animal, includes a first channel interface circuit, an output circuit, a temperature measurement circuit, and determination circuit determines whether the domestic animal is estrous based on the electrical conductivity of the body fluid of the domestic animal and the temperature within the body of the domestic animal.

### CROSS-REFERENCE TO RELATED APPLICATION

This application is a continuation of PCT/KR2021/017703 filed on Nov. 29, 2021, which is incorporated herein by reference in its entirety.

### TECHNICAL FIELD

The present invention relates to a method of determining whether a domestic animal is estrous using a biosensor implantable into the body of the domestic animal, an implantable estrous sensor for performing such determination, and a method of operating the estrous sensor.

### RELATED ART

As for recent research trends in the development of livestock information and communication technology (ICT), biometric information such as body temperature and activity is used to predict estrus, parturition, disease, etc. and is also used to perform the early detection thereof.

Overseas companies such as Bella-AG in the United States and Smaxtec in Austria have developed technologies for identifying health conditions (disease, estrus, and parturition) through signs that appear in body parts such as ears, legs, the rumen, buttocks, the vagina, and/or the like.

LiveCare, which is a Korean ICT product, proposes a method of checking the biological changes (changes in body temperature and activity) of a cow in real time by inserting a sensor into the rumen of the cow, like overseas products.

There is a research report showing that during estrus, body temperature rises above a normal temperature for more than 12 hours, resulting in a temperature in the rumen of 38.98±0.09° C.

It has been reported that as a result of measuring vaginal electrical resistance and temperature during estrus in cows, vaginal temperature increases compared to usual times, as does rumen temperature, and vaginal electrical resistance also increases due to mucus.

Meanwhile, cows after parturition have a uterine recovery period, and, even during this uterine recovery period, cows may be estrous.

A lot of research has been conducted on the phenomenon of increase in body temperature (rumen temperature, and/or vaginal temperature) during estrus. However, it is difficult to distinguish whether the behavioral pattern that appears in cows after parturition is due to estrus or due to weaning exhibiting a similar pattern (the forced separation of a suckling calf from a mother cow) because the behavioral patterns of estrus and weaning are considerably similar to each other.

Therefore, in order to increase the conception rate of cows, there is a need for a more precise method of distinguishing between estrus and weaning in weaned cows.

Many wearable or implantable sensors have been proposed to aid in the detection of estrus. For example, as prior art [1], Korean Patent No. 10-2275130 entitled “System and Method for Acquiring Estrus Information of Livestock” discloses a technology of acquiring the individual biometric information and location information of livestock through the establishment of a sensor-communication network and displaying information on display units attached to the livestock. However, the display units attached to the outsides of the bodies of livestock may be damaged by the movement of the livestock or collision between the livestock, or may be lost when the livestock bite each other.

The estrus of livestock is associated with physiological and behavioral changes. However, most sensors, including the sensor of the above prior art [1], rely on pedometers and neck/leg accelerometers to monitor increases in physical activity. With this method, it is difficult to accurately describe behavior during estrus that is caused by numerous biological factors.

Prior art [2] disclosed in the paper “Estrous detection by continuous measurements of vaginal temperature and conductivity with supervised machine learning in cattle”, Shogo Higaki et al., Theriogenology, 2018 Sep. 27 is a technology for determining whether a cow is estrous with a focus on vaginal electrical conductivity (VC) and vaginal temperature.

FIG. 1 is a diagram showing an example of the appearance and method of use of a sensor that measures electrical conductivity (EC) in the vagina of a cow in the prior art [2].

The sensor shown in FIG. 1 includes a body 100, protrusions 110 configured to maintain the sensor in the body, a rope 120 configured to pull the sensor out of the body, and a pair of ring electrodes 112 and 114 configured to measure the electrical conductivity of vaginal mucus (bovine body fluid) in the body. The sensor is inserted into the vagina of a cow. To help to understand how the sensor is maintained in the body of the cow and measures temperature and electrical conductivity, a dissected body organ of the cow and the sensor are shown together.

Prior art [3] disclosed in the paper “When is a cow in estrus? Clinical and practical aspects.” Roelofs J, Lopez-Gatius F, Hunter R, Van Eerdenburg F, Hanzen C., Theriogenology 2010; 74:327e44. (2010 August) presents research results showing that the estrus of cows is complicated to interpret because it is related to physiological and behavioral changes.

Prior art [4] disclosed in the paper “Walking activity at estrus and subsequent fertility in dairy cows.” Lopez-Gatius F, Santolaria P, Mundet I, Yaniz J., Theriogenology 2005; 63:1419e29. (2005 March) presents the results of research using a pedometer to monitor the relationship between physical activity and estrus.

The prior art [2] refers to the prior art [3] and the prior art [4], but determines whether a cow is estrous based on the electrical conductivity (VC) of mucus in the vagina of the cow and the vaginal temperature (VT) instead of the activity of the cow.

According to the prior art [2], a sensor that detects the electrical conductivity and temperature of vaginal mucus (bovine body fluid) needs to be implanted into the vagina for a long time and periodically detect electrical conductivity and temperature. Furthermore, a problem arises in that conditions that make it difficult to appropriately measure electrical conductivity and temperature occur frequently depending on an environment surrounding the sensor, such as electrical/physical characteristics within the vagina of a domestic animal, and the movement of a measurement target. When electrical conductivity and temperature data are collected only under specific conditions because it is difficult to measure electrical conductivity and temperature depending on an environment around the sensor and conditions, a problem arises in that accuracy regarding the true positive/false positive detection of estrus is significantly reduced. In order to increase the accuracy of detection of estrus by applying machine learning techniques, which have recently attracted attention, biometric data needs to be collected under various conditions. The conventional method of measuring electrical conductivity and temperature only under specific conditions is a factor that makes the accurate detection of estrus difficult.

Therefore, there is a demand for the development of a sensor that minimizes power consumption to accurately measure electrical conductivity and temperature within the vagina of a cow for a long period and is robust to an environment surrounding the sensor to measure electrical conductivity and temperature regardless of the environment surrounding the sensor.

### SUMMARY

In general, a method of using electrical conductivity in estrous sensors for livestock is to apply a low-frequency alternating current signal and measure the amplitude of a response signal. This method has a limitation in that valid data is provided only under specific measurement conditions within the body of a domestic animal (such as specific electrical characteristics surrounding a sensor within the body).

In a conventional method of measuring electrical conductivity by measuring the amplitude of a signal and calculating the real part of impedance, errors in measurement values often occur when the water content or ion concentration within mucus is excessively high or low out of an initially assumed range. This is assumed to occur because the dynamic range of an electrical conductivity sensor is not sufficiently large.

An object of the present invention is to provide an electrical conductivity sensor that may measure electrical conductivity regardless of the measurement conditions of a medium (body fluid/mucus in the body of a domestic animal)/an environment surrounding the sensor (i.e., an electrical conductivity sensor that has a large dynamic range) and also provide a sensor and method for determining the estrus of a domestic animal using the electrical conductivity sensor.

An object of the present invention is to propose an estrous sensor and method that may stably measure the temperature and electrical conductivity of a medium regardless of the current situation of the medium, thus being significantly robust even to temporary environmental changes.

An object of the present invention is to propose an estrous sensor that is non-destructive, may be used semi-permanently, provides device stability, and does not require a configuration for varying the frequency of an input electrical signal, thereby significantly reducing manufacturing costs compared to conventional estrous sensors.

An object of the present invention is to propose an estrous sensor that has a circuit and operation method capable of effectively detecting a shift in resonance frequency. Furthermore, an object of the present invention is to propose a medium monitoring sensor that does not require the process of varying the frequency of an input electrical signal, thereby shortening the time required to sense electrical conductivity in a medium.

An object of the present invention is to propose an estrous sensor that is designed to use one or two sensing electrodes depending on the embodiment and is also designed such that, when two electrodes are used, both the sensing electrodes participate in the process of measuring the electrical conductivity of a medium. Accordingly, another object of the present invention is to provide an estrous sensor that does not limit the measurement conditions/range of a medium under which characteristic parameters, such as moisture content and salts, of the medium can be monitored, may deal with a medium in various environments, and may increase the correlation between measured values over time.

According to an embodiment of the present invention, there is provided an estrous sensor that is implanted into a body of a domestic animal and detects estrus of the domestic animal. The estrous sensor may comprise: a first channel interface circuit configured to receive a first channel sensing signal having a first sensing resonance frequency from a first sensing resonance circuit connected to a first electrode in contact with body fluid of the domestic animal within the body of the domestic animal; an output circuit configured to: receive the first channel sensing signal via the first channel interface circuit; and generate a first channel output signal; and a temperature measurement circuit configured to detect temperature within the body of the domestic animal. The estrous sensor may further comprise: a determination circuit configured to: receive the first channel output signal; receive the temperature within the body of the domestic animal from the temperature measurement circuit; and determine whether the domestic animal is estrous.

The output circuit may be further configured to: generate a first channel sensing intermediate signal having a first channel differential sensing frequency, which is a difference between a first reference resonance frequency at which the first channel sensing signal has been initialized and the first sensing resonance frequency, by using a process of processing the first channel sensing signal; generate first channel sensing information based on the first channel differential sensing frequency; and output the first channel output signal having a magnitude corresponding to electrical conductivity of the body fluid of the domestic animal based on the first channel sensing information.

The determination circuit may be further configured to: detect the electrical conductivity of the body fluid of the domestic animal by the first channel output signal; and determine whether the domestic animal is estrous based on the electrical conductivity of the body fluid of the domestic animal and the temperature within the body of the domestic animal.

The estrous sensor may further comprise a second channel interface circuit configured to receive a second channel sensing signal having a second sensing resonance frequency from a second sensing resonance circuit connected to a second electrode in contact with the body fluid of the domestic animal within the body of the domestic animal.

The output circuit may be further configured to: receive the second channel sensing signal via the second channel interface circuit, and generate a second channel output signal; generate a second channel sensing intermediate signal having a second channel differential sensing frequency, which is a difference between a second reference resonance frequency, at which the second channel sensing signal has been initialized, and the second sensing resonance frequency using a process of processing the second channel sensing signal; generate second channel sensing information based on the second channel differential sensing frequency; and output the second channel output signal based on the second channel sensing information.

The determination circuit may be further configured to: receive the second channel output signal; generate channel interval information, which is a difference between the first reference resonance frequency and the second reference resonance frequency; generate channel sensing value difference information, which is a difference between the first channel sensing information and the second channel sensing information.

The determination circuit may be further configured to detect the electrical conductivity of the body fluid of the domestic animal based on a ratio between the channel sensing value difference information and the channel interval information, and determine whether the domestic animal is estrous based on the electrical conductivity of the body fluid of the domestic animal and the temperature within the body of the domestic animal.

The output circuit may be further configured to: generate first channel sensing additional information corresponding to an amplitude of the first channel sensing signal; and output a first channel additional output signal having a magnitude corresponding to the electrical conductivity of the body fluid of the domestic animal based on the first channel sensing additional information.

The determination circuit may be further configured to: cross-verify a first determination value corresponding to the electrical conductivity of the body fluid of the domestic animal detected by the first channel output signal by using a second determination value corresponding to the electrical conductivity of the body fluid of the domestic animal detected by the first channel additional output signal; generate a third determination value of the electrical conductivity of the body fluid of the domestic animal based on results of the cross-verification; and determine whether the domestic animal is estrous based on the third determination value and the temperature within the body of the domestic animal.

According to an embodiment of the present invention, there is provided an estrous sensor that is implanted into a body of a domestic animal and detects estrus of the domestic animal, the estrous sensor comprising: a first channel interface circuit configured to receive a first channel sensing signal having a first sensing resonance frequency from a first sensing resonance circuit connected to a first electrode in contact with body fluid of the domestic animal within the body of the domestic animal; and an output circuit configured to: receive the first channel sensing signal via the first channel interface circuit; and generate a first channel output signal.

The output circuit may be further configured to: generate a first channel sensing intermediate signal having a first channel differential sensing frequency, which is a difference between a first reference resonance frequency at which the first channel sensing signal has been initialized and the first sensing resonance frequency, by using a process of processing the first channel sensing signal; generate first channel sensing information based on the first channel differential sensing frequency; and output the first channel output signal having a magnitude corresponding to electrical conductivity of the body fluid of the domestic animal based on the first channel sensing information.

The output circuit may be further configured to transfer the first channel output signal to a determination circuit configured to detect temperature within the body of the domestic animal by a temperature measurement circuit so that the determination circuit detects the electrical conductivity of the body fluid of the domestic animal by the first channel output signal and determines whether the domestic animal is estrous based on the electrical conductivity of the body fluid of the domestic animal and the temperature within the body of the domestic animal.

According to an embodiment of the present invention, there is provided an estrus determination method using an estrous sensor that is implanted into a body of a domestic animal and detects estrus of the domestic animal, the estrus determination method comprising: receiving a first channel sensing signal having a first sensing resonance frequency from a first sensing resonance circuit connected to a first electrode in contact with body fluid of the domestic animal within the body of the domestic animal; detecting temperature within the body of the domestic animal; generating a first channel sensing intermediate signal having a first channel differential sensing frequency, which is a difference between a first reference resonance frequency at which the first channel sensing signal has been initialized and the first sensing resonance frequency, by using a process of processing the first channel sensing signal; generating first channel sensing information based on the first channel differential sensing frequency; and generating the first channel output signal having a magnitude corresponding to electrical conductivity of the body fluid of the domestic animal based on the first channel sensing information.

The estrus determination method may further comprise: detecting the electrical conductivity of the body fluid of the domestic animal by the first channel output signal; and determining whether the domestic animal is estrous based on the electrical conductivity of the body fluid of the domestic animal and the temperature within the body of the domestic animal.

The estrus determination method may further comprise, before detecting the electrical conductivity of the body fluid of the domestic animal: receiving a second channel sensing signal having a second sensing resonance frequency from a second sensing resonance circuit connected to a second electrode in contact with the body fluid of the domestic animal within the body of the domestic animal; receiving the second channel sensing signal via the second channel interface circuit, and generating a second channel output signal; generating a second channel sensing intermediate signal having a second channel differential sensing frequency, which is a difference between a second reference resonance frequency, at which the second channel sensing signal has been initialized, and the second sensing resonance frequency using a process of processing the second channel sensing signal; generating second channel sensing information based on the second channel differential sensing frequency; and generating the second channel output signal based on the second channel sensing information.

The detecting the electrical conductivity of the body fluid of the domestic animal may comprise: generating channel interval information, which is a difference between the first reference resonance frequency and the second reference resonance frequency; generating channel sensing value difference information, which is a difference between the first channel sensing information and the second channel sensing information; and detecting the electrical conductivity of the body fluid of the domestic animal based on a ratio between the channel sensing value difference information and the channel interval information.

The estrus determination method further comprising, before detecting the electrical conductivity of the body fluid of the domestic animal, generating first channel sensing additional information corresponding to an amplitude of the first channel sensing signal and outputting a first channel additional output signal having a magnitude corresponding to the electrical conductivity of the body fluid of the domestic animal based on the first channel sensing additional information.

The detecting the electrical conductivity of the body fluid of the domestic animal comprises: cross-verifying a first determination value corresponding to the electrical conductivity of the body fluid of the domestic animal detected by the first channel output signal by using a second determination value corresponding to the electrical conductivity of the body fluid of the domestic animal detected by the first channel additional output signal; and generating a third determination value of the electrical conductivity of the body fluid of the domestic animal as a final determination value of the electrical conductivity of the body fluid of the domestic animal based on results of the cross-verification.

According to an embodiment of the present invention, there is provided an estrus determination method using an estrous sensor that is implanted into a body of a domestic animal and detects estrus of the domestic animal, the estrus determination method comprising: receiving a first channel sensing signal having a first sensing resonance frequency from a first sensing resonance circuit connected to a first electrode in contact with body fluid of the domestic animal within the body of the domestic animal; generating a first channel sensing intermediate signal a having first channel differential sensing frequency, which is a difference between a first reference resonance frequency at which the first channel sensing signal has been initialized and the first sensing resonance frequency, by using a process of processing the first channel sensing signal; generating first channel sensing information based on the first channel differential sensing frequency; and outputting the first channel output signal having a magnitude corresponding to electrical conductivity of the body fluid of the domestic animal based on the first channel sensing information.

The estrus determination method may further comprise transferring the first channel output signal to an estrous determination circuit so that the estrous determination circuit detects the electrical conductivity of the body fluid of the domestic animal by the first channel output signal and determines whether the domestic animal is estrous based on the electrical conductivity of the body fluid of the domestic animal and the temperature within the body of the domestic animal.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a diagram showing an example of the appearance and method of use of a sensor that measures electrical conductivity (EC) in the vagina of a cow in the prior art;

FIG. 2 is a diagram the configuration and operating principle of an estrous sensor 200 according to an embodiment of the present invention;

FIG. 3 is a diagram illustrating the configuration and operating principle of an estrous sensor 300 according to an embodiment of the present invention;

FIG. 4 is a diagram illustrating the internal configuration and operating principle of an electrical conductivity sensing interface circuit 210 in an estrous sensor 200 according to an embodiment of the present invention;

FIG. 5 is a diagram illustrating the internal configuration and operating principle of an electrical conductivity sensing interface circuit 310 in an estrous sensor according to an embodiment of the present invention;

FIG. 6 is a diagram showing the interaction between the first sensing resonance circuit 410 and first channel interface circuit 420 of the estrous sensor 200 according to an embodiment of the present invention;

FIG. 7 is a diagram showing the interaction between the second sensing resonance circuit 412 and second channel interface circuit 422 of the estrous sensor 200 according to an embodiment of the present invention;

FIG. 8 shows the configuration of the output circuit 430 of the electrical conductivity sensing interface circuit 210 according to an embodiment of the present invention;

FIG. 9 is an operational flowchart illustrating an embodiment of a method of operating the electrical conductivity sensing interface circuit of FIGS. 2 and 4;

FIG. 10 is an operational flowchart illustrating an embodiment of a method of operating the estrous sensor of FIG. 2;

FIG. 11 is an operational flowchart illustrating step S930 of FIGS. 9 and 10 in detail;

FIG. 12 is an operational flowchart illustrating step S940 of FIGS. 9 and 10 in detail;

FIG. 13 is a diagram of an equivalent circuit used to illustrate the operating principle of the estrous sensor 200 according to an embodiment of the present invention; and

FIG. 14 is a diagram illustrating the operating principle by which the estrous sensor 200 according to an embodiment of the present invention determines electrical conductivity.

### DETAILED DESCRIPTION

Other objects and features of the present invention in addition to the above-described objects will be apparent from the following description of embodiments to be given with reference to the accompanying drawings.

The embodiments of the present invention will be described in detail below with reference to the accompanying drawings. In the following description, when it is determined that a detailed description of a known component or function may unnecessarily make the gist of the present invention obscure, it will be omitted.

Relational terms such as first, second, and the like may be used for describing various elements, but the elements should not be limited by the terms. These terms are only used to distinguish one element from another. For example, a first component may be named a second component without departing from the scope of the present disclosure, and the second component may also be similarly named the first component. The term “and/or” means any one or a combination of a plurality of related and described items.

When it is mentioned that a certain component is “coupled with” or “connected with” another component, it should be understood that the certain component is directly “coupled with” or “connected with” to the other component or a further component may be disposed therebetween. In contrast, when it is mentioned that a certain component is “directly coupled with” or “directly connected with” another component, it will be understood that a further component is not disposed therebetween.

The terms used in the present disclosure are only used to describe specific exemplary embodiments, and are not intended to limit the present disclosure. The singular expression includes the plural expression unless the context clearly dictates otherwise. In the present disclosure, terms such as ‘comprise’ or ‘have’ are intended to designate that a feature, number, step, operation, component, part, or combination thereof described in the specification exists, but it should be understood that the terms do not preclude existence or addition of one or more features, numbers, steps, operations, components, parts, or combinations thereof.

Unless otherwise defined, all terms (including technical and scientific terms) used herein have the same meaning as commonly understood by one of ordinary skill in the art to which this disclosure belongs. Terms that are generally used and have been in dictionaries should be construed as having meanings matched with contextual meanings in the art. In this description, unless defined clearly, terms are not necessarily construed having formal meanings.

Some of the contents disclosed in these related art documents are related to the objects to be achieved by the present invention, and some of the solutions adopted by the present invention are applied to these related art documents in the same manner.

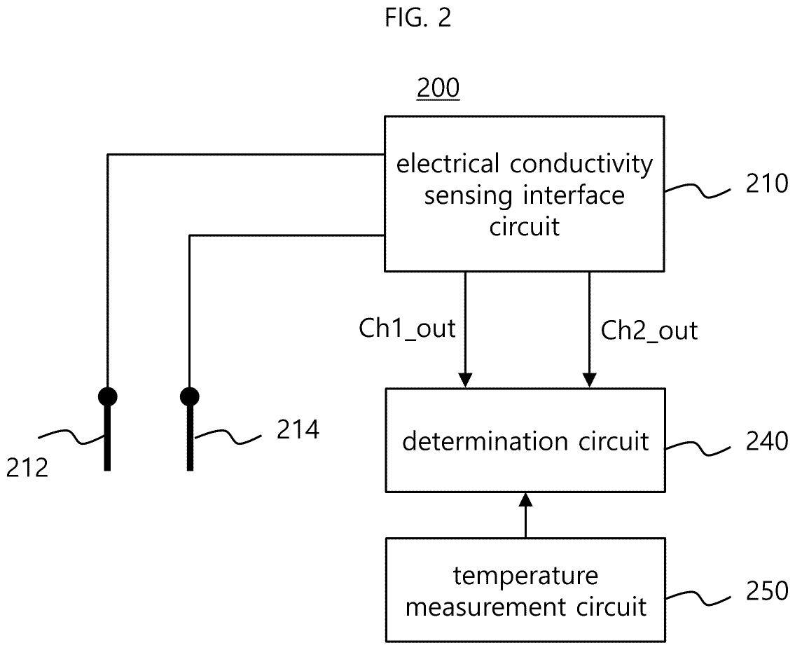

In addition to the above objects, other objects and features of the present invention will be clearly revealed through the description of embodiments taken in conjunction with the accompanying drawings. Embodiments of the present invention will be described in detail with reference to the accompanying drawings. In the following description of the present invention, when it is determined that a detailed description of a related known configuration or function may obscure the gist of the present invention, the detailed description will be omitted. Hereinafter, an electrical conductivity sensing interface circuit, an estrous sensor, and methods of operating the same according to some embodiments of the present invention will be described in detail with reference to FIGS. 2 to 14.

FIG. 2 is a diagram illustrating the configuration and operating principle of an estrous sensor 200 according to an embodiment of the present invention.

The estrous sensor 200 according to the present embodiment includes a first electrode 212 configured to come into contact with a medium, a second electrode 214 configured to come into contact with the medium, an electrical conductivity sensing interface circuit 210, and a determination circuit 240. The embodiment of FIG. 2 may further include a temperature measurement circuit 250.

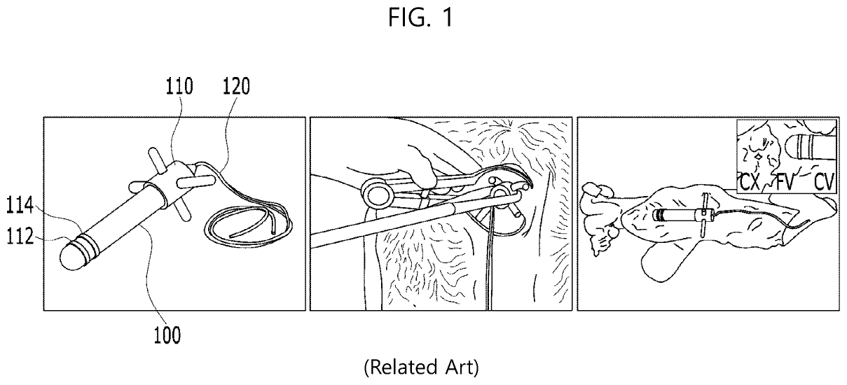

Although not shown in FIG. 2, the estrous sensor 200 may include a case including a printed circuit board, an electronic circuit, etc. The external shapes of the external case of the estrous sensor 200 and the two sensing electrodes 212 and 214 may be modified to fit the purpose of the present invention by referring to the configurations shown in FIG. 1, which is a prior art.

Meanwhile, although the pair of ring electrodes 112 and 114 are shown as constituting one channel for measuring electrical conductivity in FIG. 1, a separate common ground electrode may be exposed to the outside or only the sensing electrodes 212 and 214 for two channels may be exposed to the outside without a common ground electrode exposed to the outside in the present invention. This is the difference that arises because the electrical conductivity measurement method of the present invention is different from the electrical conductivity measurement method of the prior art.

For convenience of description, it is assumed that the two sensing electrodes 212 and 214 are identified as the first electrode 212 and the second electrode 214.

The first electrode 212 is involved in a first channel sensing operation, and the second electrode 214 is involved in a second channel sensing operation. Both the first channel sensing operation and the second channel sensing operation may be applied to measure the electrical conductivity of a medium (body fluid in the body of a domestic animal, e.g., mucus in the vagina of a cow).

Prior arts, including the prior art [1] disclosed in Korean Patent No. 10-2275130 and the prior art [2] disclosed in the paper “Estrous detection by continuous measurements of vaginal temperature and conductivity with supervised machine learning in cattle” (Shogo Higaki et al.), are configured to apply an alternating current signal between two electrodes to measure the electrical conductivity of a medium, detect a change in impedance by detecting the amplitude of a received signal, and measure electrical conductivity based on the change in impedance.

The two electrodes shown in FIG. 2 of the present application are electrodes assigned to channels that function independently of each other, and a common ground electrode (not shown) may be exposed to the outside, but is not necessarily exposed to the outside. In the embodiment of FIG. 2, the two channel electrodes may individually and independently detect changes in impedance, and the electrical conductivity of a medium may be determined based on a combination of these changes. In another embodiment, electrical conductivity may be determined in such a manner that two channels contribute to the determination of the electrical conductivity of a medium together. These embodiments of the present invention are differentiated from the prior arts for measuring electrical conductivity.

The domestic animal may be an animal that has estrus and weaning periods. For example, a cow, a goat, a pig, etc. may be subject to the present invention. In the case of a cow, the vulva is a sensitive area, so that the cow may become unstable due to stimulation when a sensor is brought out from the vulva, or the cow may be bitten by another cow, with the result that a sensor that is introduced into the body is required.

Furthermore, the part of the sensor inserted into the vagina needs to have protrusions or bends to be maintained within the vagina, and needs to be designed such that it does not easily fall out to the outside.

The part of the sensor exposed out of the body needs to be made as small/thin/flexible as possible in order not to irritate a domestic animal.

The sensor is fabricated to be disposable for the purpose of preventing disease, and a battery is required for the sensor to operate within the body. The battery requires a lifespan of approximately two years, and it is appropriate to design and implement the battery so that it can be used from estrus through pregnancy to parturition.

The prior arts generally distinguish estrus and weaning periods based on the activity of livestock. However, livestock generally go through a weaning process two months after parturition. Even during this process, their activity level may become significantly high. In other words, it is difficult to distinguish based on activity alone whether a high activity level is a sign for true estrus or a temporary increase in activity experienced during a weaning process.

In response to this, there are previous studies that distinguish estrus and weaning periods by measuring activity (measured outside the body) and intra-body temperature together. The present invention proposes an estrous sensor that determines estrus and weaning periods by being implanted into the body in order not to stimulate a domestic animal outside the body and then measuring both electrical conductivity and temperature within the body.

The estrous sensor based on electrical conductivity and body temperature according to the present invention may be used alone, or may be combined with a means of measuring activity outside the body using the conventional method and more accurately determine an estrus period.

The estrous sensor of the present invention may measure electrical conductivity and body temperature within the body, may measure activity using a conventional inertial sensor (a pedometer, an acceleration sensor, and/or the like), may accurately distinguish between estrus and weaning periods by considering all the body electrical conductivity, the body temperature and the activity, and may effectively determine an estrus period.

The estrous sensor of the present invention may minimize the area exposed out of the body, or may provide a sensor that is virtually not exposed out of the body.

The part of the estrous sensor that is exposed out of the body is a thin thread/string connected to the end of the sensor, and this may be a part intended to be used to pull the sensor out of the body.

Protrusions that secure the estrous sensor onto the body are formed on the estrous sensor, so that even when the exposed thread/string is simply pulled outside the body, the sensor is not easily discharged to the outside. As long as the sensor is not discharged from the body, there is no damage to the sensor and also there is no influence on the operation of the sensor.

To discharge the sensor from the body, a pre-arranged signal may be transmitted to the sensor through wireless communication, and then the protrusions or the like used to secure the sensor onto the body may be transformed into a state that allows the sensor to be easily discharged from the body.

The sensor needs to operate within the body for a long period, and thus a waterproof function is required for the sensor.

For communication with the outside, a variety of general wireless communication methods may be applied.

A sensor may be combined with the estrous sensor and measure activity, or the estrous sensor itself may measure activity. A variety of methods, such as a method of measuring the activity of an individual using an inertial sensor or an acceleration sensor and a method of measuring the activity of a group using an inertial sensor, an acceleration sensor or a camera, may be used to measure activity.

The electrical conductivity sensing interface circuit 210 may receive a first channel sensing signal resulting from a first channel sensing operation between the first electrode 212 and the common ground and output a first channel output signal Ch1_out, and may receive a second channel sensing signal resulting from a second channel sensing operation between the second electrode 214 and the common ground and output a second channel output signal Ch2_out. The electrical conductivity sensing interface circuit 210 may be considered a type of readout integrated circuit (ROIC). In this case, the common ground may be exposed to the outside separately in addition to the two electrodes 212 and 214, or the electrical conductivity sensing interface circuit 210 and the determination circuit 240 may be connected in common to the common ground inside the circuit without exposure to the outside.

The determination circuit 240 may receive the first channel output signal Ch1_out and the second channel output signal Ch2_out and measure the electrical conductivity of the medium. Furthermore, the determination circuit 240 may determine whether a domestic animal is estrous based on the electrical conductivity of the medium and the temperature of the medium measured by the temperature measurement circuit 250.

A logic condition or algorithm by which the determination circuit 240 determines whether a domestic animal is estrous based on the electrical conductivity of a medium (body fluid/mucus within the body/vagina of a domestic animal/a cow) and the temperature within the medium is covered in detail by prior arts, such as the prior art [2] disclosed in the paper “Estrous detection by Continuous measurements of vaginal temperature and conductivity with supervised machine learning in cattle”, Shogo Higaki et al., Theriogenology, 2018 Sep. 27. The configurations disclosed in the above prior art document, i.e., the configuration for determining whether a domestic animal is estrous based on electrical conductivity and temperature, the configuration for improving the accuracy of determination of the estrus of a domestic animal based on electrical conductivity and temperature measurement data through supervised learning using an artificial neural network, etc., may be incorporated into the configurations of the present invention to the extent that they meet the purpose of the present invention. Detailed descriptions thereof will be omitted because they can be easily understood by those skilled in the art through the above prior document.

The process of measuring the electrical conductivity of a medium (body fluid) is performed based on the frequency difference between a sensing resonance frequency and a reference resonance frequency in each of the channels. Finally, the electrical conductivity of the medium (body fluid) may be determined based on a combination of a resonance frequency shift value and change in the amplitude measured in each of the channels. Alternatively, the electrical conductivity of the medium (body fluid) may be determined by prioritizing one of two or more channels and using the resonance frequency shift value and change in amplitude of the prioritized channel.

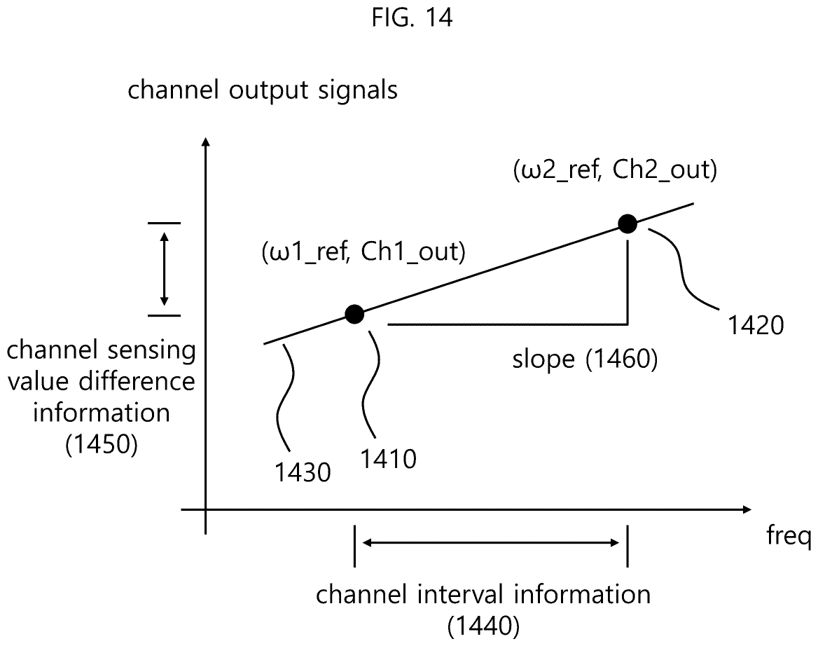

The process of measuring the electrical conductivity of the medium may be performed using the difference between the resonance frequency shift values obtained in the individual channels. In this case, the individual channels may be initialized based on different reference resonance frequencies. In other words, the reference resonance frequencies at which the individual channels have been initialized are different from each other, and the resonance frequency shift values measured at different reference frequencies may include both the contribution part of a reactance change and the contribution part of a resistance component attributable to the electrical characteristics of the medium. Using these characteristics, the electrical conductivity of the medium may be determined using different channel measurement values (resonance frequency shift values) measured at different reference frequencies. Details related to this will be described below with reference to FIG. 14.

In an embodiment of the present invention, one of the first electrode 212 and the second electrode 214 to which a high frequency will be applied and the other of the first electrode 212 and the second electrode 214 to which a low frequency will be applied may be determined and fixed in advance. In another embodiment of the present invention, high and low frequencies applied to the first electrode 212 and the second electrode 214 may be interchanged with each other by changing electrical positions using a MUX circuit.

The mutual verification and calibration between the channel electrodes and the output signals may be performed by comparing and verifying a first operation of applying a high frequency to the first electrode 212 and a low frequency to the second electrode 214 and a second operation of applying a low frequency to the first electrode 212 and a high frequency to the second electrode 214 using a MUX circuit. This process may further enhance the calibration between the channel electrodes.

In an embodiment of the present invention, the resonance frequency of an initialized alternating current signal applied to each of the first electrode 212 and the second electrode 214 may be fixed by electrical elements R, L, and C on a printed circuit board.

In another embodiment of the present invention, the reference resonance frequency may be changed by selectively connecting capacitors and/or inductors having different values using a MUX circuit. When the reference resonance frequency is changed in this manner, calibration for each of the first electrode 212 and the second electrode 214 may be performed by comparing and verifying output signals measured from the electrode 212 or 214.

In this case, the electrical conductivity of the medium may be determined based on the ratio between channel interval information and channel sensing value difference information. The channel interval information may correspond to the difference between the first reference resonance frequency ω1_ref at which the first channel sensing signal has been initialized and the second reference resonance frequency ω2_ref at which the second channel sensing signal has been initialized. The channel sensing value difference information may correspond to the difference between first channel sensing information and second channel sensing information, the first channel sensing information may be obtained from the first channel output signal Ch1_out, and the second channel sensing information may be obtained from the second channel output signal Ch2_out.

Although not shown in FIG. 2, the electrical conductivity sensing interface circuit 310 may include four resonance circuits (sensing resonance circuits for the two channels and reference resonance circuits for the two channels).

The determination circuit 240 may be implemented by, e.g., a microprocessor or a microcontroller unit (MCU), and may be combined with a wired or wireless communication module and transmit measurement results to an external server or database so that the measurement results are stored in the server or database.

The electrical conductivity sensing interface circuit 210, the determination circuit 240, and the temperature measurement circuit 250 may be implemented as respective integrated circuits (ICs) and disposed on one printed circuit board. In another embodiment, at least two of the electrical conductivity sensing interface circuit 210, the determination circuit 240, and the temperature measurement circuit 250 may be embedded in one integrated circuit (IC).

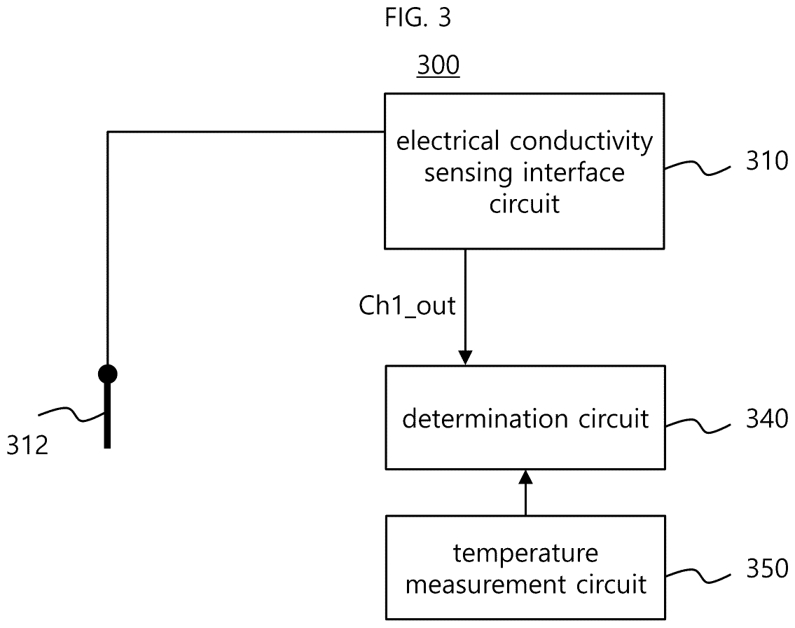

FIG. 3 is a diagram illustrating the configuration and operating principle of an estrous sensor 300 according to an embodiment of the present invention. FIG. 3 shows an embodiment in which only a first electrode 312 corresponding to one channel is exposed to the outside. Like in the embodiment of FIG. 2, a common ground may be exposed to the outside, or individual circuits may be connected in common to a common ground only within a circuit.

The estrous sensor 300 according to an embodiment of the present invention includes a first electrode 312 configured to come into contact with a medium, an electrical conductivity sensing interface circuit 310, and a determination circuit 340. The estrous sensor 300 may further include a temperature measurement circuit 350.

The electrical conductivity sensing interface circuit 310 may receive a first channel sensing signal resulting from a first channel sensing operation between the first electrode 212 and the common ground within the circuit and output a first channel output signal Ch1_out. The electrical conductivity sensing interface circuit 310 may be considered a type of ROIC.

The determination circuit 340 may receive the first channel output signal Ch1_out and measure the electrical conductivity of a medium. Furthermore, the determination circuit 340 may determine whether a domestic animal is estrous based on the electrical conductivity of the medium m and the temperature of the medium measured by the temperature measurement circuit 350.

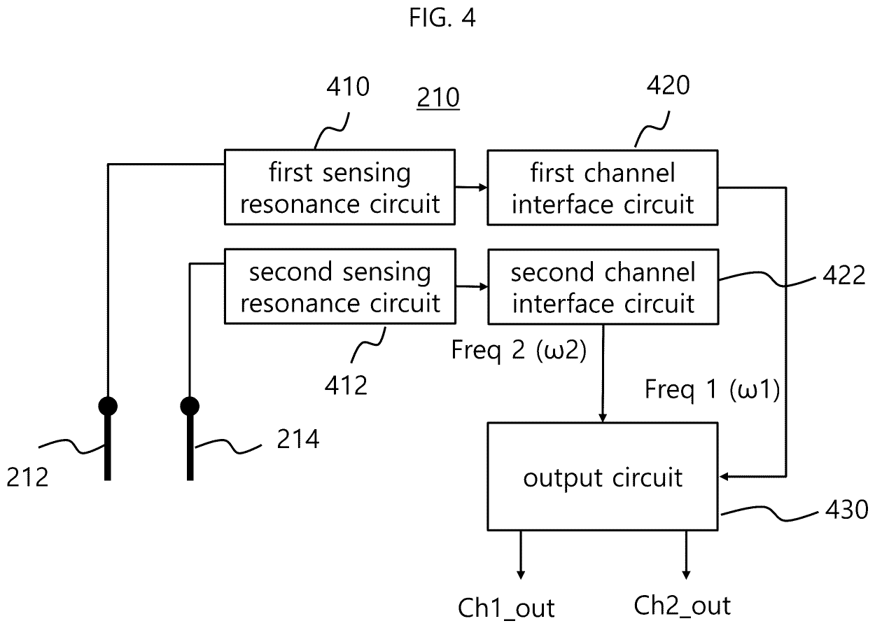

FIG. 4 is a diagram illustrating the internal configuration and operating principle of an electrical conductivity sensing interface circuit 210 in an estrous sensor 200 according to an embodiment of the present invention.

The electrical conductivity sensing interface circuit 210 according to the present embodiment includes: a first channel interface circuit 420 configured to receive a first channel sensing signal having a first sensing resonance frequency ω1 from a first sensing resonance circuit 410 connected to a first electrode 212 in contact with a medium; a second channel interface circuit 422 configured to receive a second channel sensing signal having a second sensing resonance frequency ω2 from a second sensing resonance circuit 412 connected to a second electrode 214 in contact with the medium; and an output circuit 430 configured to receive the first channel sensing signal via the first channel interface circuit 420, receive the second channel sensing signal via the second channel interface circuit 422, and generate a first channel output signal Ch1_out and a second channel output signal Ch2_out.

In this case, the output circuit 430 generates first channel sensing information based on the first sensing resonance frequency ω1 using the first channel sensing signal, generates second channel sensing information based on the second sensing resonance frequency ω2 using the second channel sensing signal, outputs the first channel output signal Ch1_out based on the first channel sensing information, and outputs the second channel output signal Ch2_out based on the second channel sensing information.

In this case, the electrical conductivity of the medium may be independently determined in a first channel based on a combination of a change in the amplitude of the first channel sensing signal and the first channel output signal Ch1_out (corresponding to a shift in the resonance frequency of the first channel sensing signal), and may be independently determined in a second channel based on a combination of a change in the amplitude of the second channel channel sensing signal and the second output signal Ch2_out (corresponding to a shift in the resonance frequency of the second channel sensing signal). The electrical conductivity of the medium may be determined based on a combination of the electrical conductivity determination values of the first and second channels. In this case, the electrical conductivity of the medium may be determined based on at least one of the electrical conductivity of the first channel and the electrical conductivity of the second channel, or may be determined based on a combination of the electrical conductivity of the first channel and the electrical conductivity of the second channel. A simple example of a combination of the electrical conductivity of the first channel and the electrical conductivity of the second channel is the arithmetic mean thereof. Alternatively, a weight may be assigned to either the first channel or the second channel according to the results of the calibration, and a representative value reflecting the weight therein may be generated.

In another embodiment, the electrical conductivity of the medium may be determined based on the ratio between channel interval information and channel sensing value difference information. The channel interval information may be the difference between the first reference resonance frequency ω1_ref at which the first channel sensing signal has been initialized and the second reference resonance frequency ω2_ref at which the second channel sensing signal has been initialized. The channel sensing value difference information may be the difference between the first channel sensing information and the second channel sensing information.

In this case, the first channel interface circuit 420 may receive a first channel reference signal having a first reference resonance frequency ω1_ref from a first reference resonance circuit, and the second channel interface circuit 422 may receive a second channel reference signal having a second reference resonance frequency ω2_ref from a second reference resonance circuit.

In this case, the output circuit 430 may generate first channel sensing information based on the difference between the first sensing resonance frequency ω1 and the first reference resonance frequency ω1_ref through the process of processing the first channel sensing signal and the first reference signal, and may generate second channel sensing information based on the difference between the second sensing resonance frequency ω2 and the second reference resonance frequency ω2_ref through the process of processing the second channel sensing signal and the second channel reference signal.

In this case, the output circuit 430 may include: a signal processing circuit configured to generate a first channel differential signal having a first channel differential resonance frequency corresponding to the difference between the first sensing resonance frequency ω1 and the first reference resonance frequency ω1_ref through the process of processing the first channel sensing signal and the first channel reference signal and generate a second channel differential signal having a second channel differential resonance frequency corresponding to the difference between the second sensing resonance frequency ω2 and the second reference resonance frequency ω2_ref through the process of processing the second channel sensing signal and the second channel reference signal; and a signal conversion circuit configured to generate a first channel output signal Ch1_out having a magnitude proportional to the first channel differential resonance frequency and generate a second channel output signal Ch2_out having a magnitude proportional to the second channel differential resonance frequency.

In this case, the output circuit 430 may provide a medium biosignal monitoring function by which changes in the electrical conductivity of the medium over time are represented by changes in the first channel sensing information and the second channel sensing information over time.

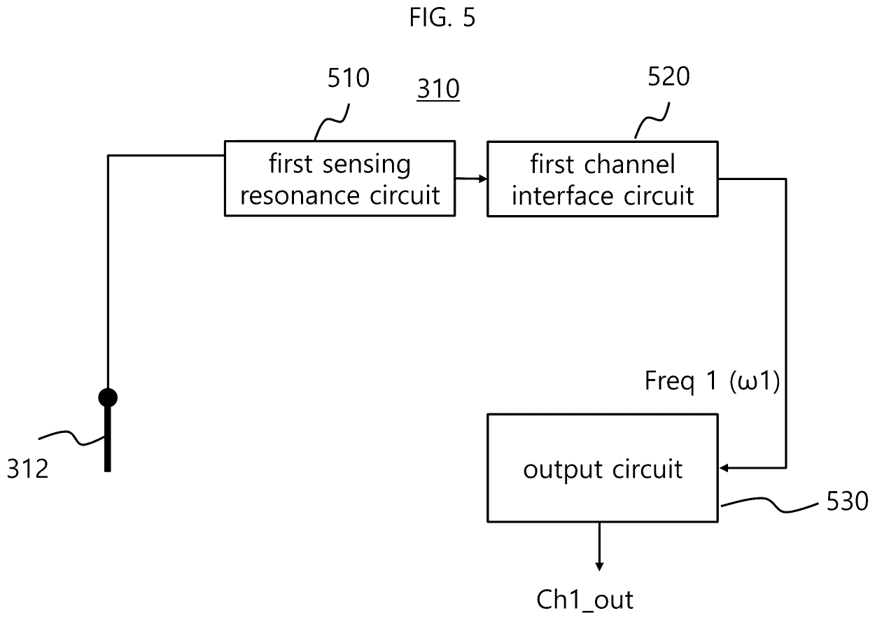

FIG. 5 is a diagram illustrating the internal configuration and operating principle of an electrical conductivity sensing interface circuit 310 in an estrous sensor according to an embodiment of the present invention.

The electrical conductivity sensing interface circuit 310 according to the present embodiment may include: a first channel interface circuit 520 configured to receive a first channel sensing signal having a first sensing resonance frequency ω1 from a first sensing resonance circuit 510 connected to a first electrode 312 in contact with a medium; and an output circuit 530 configured to receive a first channel sensing signal via a first channel interface circuit 520 and generate a first channel output signal Ch1_out.

The first sensing resonance circuit 510 of FIG. 5 operates similarly to the first sensing resonance circuit 410 of FIG. 4, the first channel interface circuit 420 of FIG. 5 operates similarly to the first channel interface circuit 520 of FIG. 4, the output circuit 530 of FIG. 5 operates similarly to the output circuit 430 of FIG. 4, and the first electrode 212 of FIG. 5 operates similarly to the first electrode 312 of FIG. 4. Accordingly, redundant descriptions thereof will be omitted.

Referring to FIGS. 2 to 5, each of the estrous sensors 200 and 300 of FIGS. 2 and 3 may perform temperature compensation on the biological signal measurement value of the medium by using the temperature measured by the temperature measurement circuit 250 or 350. The measured temperature is used for the determination of estrus, but may also be used for the compensation of a biosignal measurement value.

Each of the determination circuits 240 and 340 may obtain a first determination value for the measurement of the electrical conductivity of the medium before the compensation of temperature. The determination circuit 240 or 340 may generate a second determination value for the measurement of the electrical conductivity of the medium by compensating the first determination value based on the temperature measured by the temperature sensor. In this case, although the first and second determination values are introduced for convenience of description, each channel sensing signal before being received by the determination circuit 240 or 340 may be compensated in the output circuit 430 or 530 and then provided to the determination circuit 240 or 340. In this case, each of the output circuits 430 and 530 may generate a second measured value by applying determination criteria, based on the temperature measured by the temperature sensor, to a first measured value before compensation. Since it is known that the electrical conductivity of the medium is influenced by temperature and thus the measured value thereof varies, a second measured value or second determination value for the temperature-compensated electrical conductivity of the medium may be generated by applying temperature-based electrical conductivity determination criteria to the first measurement value or first determination value before temperature compensation in the output circuit 430 or 530 and/or the determination circuit 240 or 340.

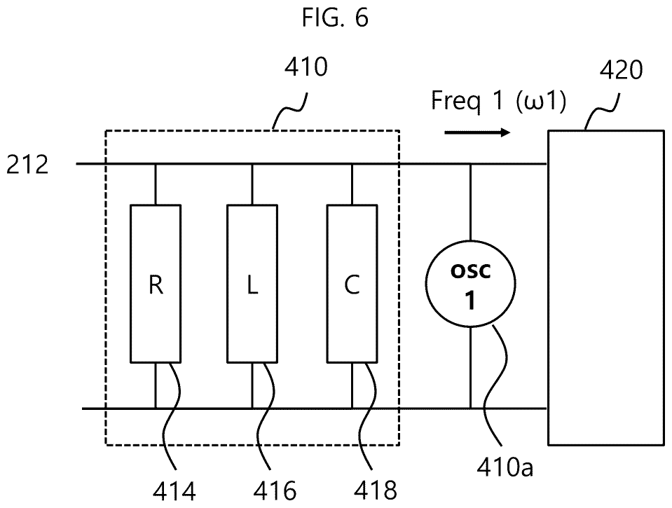

FIG. 6 is a diagram showing the interaction between the first sensing resonance circuit 410 and first channel interface circuit 420 of the estrous sensor 200 according to an embodiment of the present invention.

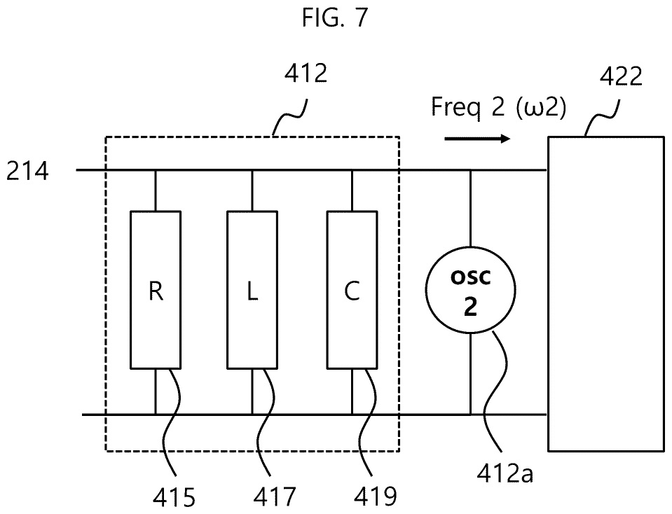

FIG. 7 is a diagram showing the interaction between the second sensing resonance circuit 412 and second channel interface circuit 422 of the estrous sensor 200 according to an embodiment of the present invention.

Referring to FIGS. 6 and 7 together, a first oscillator circuit 410a excites an alternating current signal of a first reference resonance frequency ω1_ref in the first sensing resonance circuit 410, and a second oscillator circuit 412b excites an alternating current signal of a second reference resonance frequency ω2_ref in the second sensing resonance circuit 412.

Before the electrodes 212 and 214 of the estrous sensor 200 come into contact with a medium, i.e., when the first sensing resonance circuit 410 or the second sensing resonance circuit 412 has been initialized, the first reference resonance frequency ω1_ref is maintained in the first sensing resonance circuit 410, and the second reference resonance frequency ω2_ref is maintained in the second sensing resonance circuit 412.

When the electrodes 212 and 214 of the estrous sensor 200 come into contact with a medium, each of the first sensing resonance circuit 410 and the second sensing resonance circuit 412 is influenced based on the ion concentration and electrical conductivity of the medium. When the influence of the ion concentration and electrical conductivity of the medium is represented by an equivalent circuit, it may be considered combinations of a parasitic capacitance Cx and a parasitic resistance component Rx with the first and second sensing resonance circuits 410 and 412, respectively.

The alternating current (AC) signal excited in each of the first sensing resonance circuit 410 and the second sensing resonance circuit 412 is influenced by the ion concentration and electrical conductivity of the medium, thereby forming an AC signal having a new resonance frequency. In this case, when the alternating current signal formed in the first sensing resonance circuit 410 is referred to as a first channel sensing signal and the alternating current signal formed in the second sensing resonance circuit 412 is referred to as a second channel sensing signal, the resonance frequency of the first channel sensing signal may be referred to as a first sensing resonance frequency ω1 and the resonance frequency of the second channel sensing signal may be referred to as a second sensing resonance frequency ω2.

The resonance frequency of the resonance circuit of each channel is shifted from the initialized reference resonance frequency of each channel due to the influence of the ion concentration and electrical conductivity of the medium, and thus a sensing resonance frequency is formed. Accordingly, sensing information for the ion concentration and electrical conductivity of the medium may be obtained by quantitatively detecting a shift in the resonance frequency of each channel.

In this case, although there may be employed a method of detecting the reference resonance frequency and sensing resonance frequency of each channel and calculating the difference therebetween, the reference resonance frequency of the resonance circuit changes in real time due to the influence of temperature and/or the like. Accordingly, this method may not accurately detect real-time changes in the environment of the medium being measured.

Therefore, in the present invention, separate reference resonance circuits are disposed for respective channels, and there is employed a method of obtaining an alternating current signal, whose frequency is the difference between the sensing resonance frequency of each channel and the reference resonance frequency, by processing the sensing signal of each channel and the reference signal of the reference resonance circuit, instead of a method of calculating and subtracting the sensing resonance frequency and reference resonance frequency of each channel.

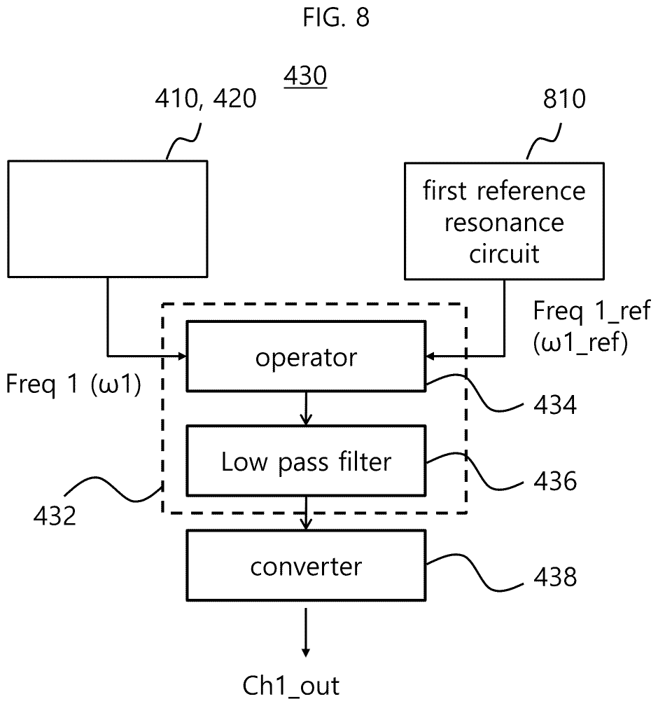

In other words, a first channel intermediate signal having a frequency ω1-ω1_ref is generated in the first channel, and a second channel intermediate signal having a frequency ω2-ω2_ref is generated in the second channel. First channel sensing information may be obtained from the first channel intermediate signal, and second channel sensing information may be obtained from the second channel intermediate signal. Details related to this will be described with reference to FIG. 8 below.

The first sensing resonance circuit 410 may include a resistor 414, an inductor 416, and a capacitor 418. The resistor 414, the inductor 416, and the capacitor 418 are merely shown as an equivalent circuit. In reality, the first sensing resonance circuit 410 may be implemented as a combination of more complex passive elements.

The second sensing resonance circuit 412 may include a resistor 415, an inductor 417, and a capacitor 419. Redundant descriptions present between the first sensing resonance circuit 410 and the second sensing resonance circuit 412 will be omitted.

In a fixed manner, the first electrode 212 and the first sensing resonance circuit 410 may be connected to each other and the second electrode 214 and the second sensing resonance circuit 412 may be connected to each other. When connection is performed via a MUX circuit, the first electrode 212 and the second sensing resonance circuit 412 may be connected to each other and the second electrode 230 and the first sensing resonance circuit 410 may be connected to each other in a crossing manner. The calibration of each channel may be possible through this cross-connection as described above.

One or more additional resistors, inductors, and capacitors may be selectively connected to each of the first and second sensing resonance circuits 410 and 412 via another MUX circuit, and thus the resonance frequency may be adjusted. The calibration of each channel may be possible through this adjustment, as described above. Furthermore, it will be readily understood by those skilled in the art that in the case where the resonance frequency of each channel is adjusted, there is no problem with a resonance frequency shift detection function only when the sensing resonance circuit and reference resonance circuit of each channel are adjusted equally.

In FIG. 6, there is shown an embodiment in which the first oscillator circuit 410a is disposed between the first sensing resonance circuit 410 and the first channel interface circuit 420. In another embodiment of the present invention, the first oscillator circuit 410a may be included in any one of the first sensing resonance circuit 410, the first channel interface circuit 420, and the output circuit 430. The second oscillator circuit 412a of FIG. 7 may also be changed in the same manner.

Furthermore, the inductors 416 and 417 may each have a coil form, but may each be implemented in the form of a semiconductor pattern having a controllable inductance. In addition to the resistors 414 and 415 shown in FIGS. 6 and 7, a resistor R′ (not shown) may be additionally disposed for balancing between other circuits.

Each of the oscillator circuits 410a and 412a for sensing may be implemented as a push-pull type oscillator circuit. An oscillator circuit having a function equivalent to that of the first oscillator circuit 410a may be connected to a first reference resonance circuit having characteristics equivalent to those of the first sensing resonance circuit 410 (ω1_ref). An oscillator circuit having a function equivalent to that of the second oscillator circuit 412a may be connected to a second reference resonance circuit having characteristics equivalent to those of the second sensing resonance circuit 412 (ω2_ref).

Four resonance circuits may be implemented as passive elements that are disposed on a printed circuit board outside the electrical conductivity sensing interface circuit 310. The oscillator circuits 410a and 412a for sensing and the equivalent reference oscillator circuits may be disposed as a single integrated circuit within each of the electrical conductivity sensing interface circuits 210 and 310 by considering spatial disposition and current consumption. This disposition of oscillator circuits does not require additional active elements, which may be advantageous for small size and low-power configuration.

When the oscillator circuits are disposed inside each of the electrical conductivity sensing interface circuits 210 and 310, which is one chip, the oscillator circuits having equivalent performance are disposed close to each other in terms of layout and are formed of the same type of elements. Accordingly, measurement errors attributable to process variations may be reduced, so that the ability to detect shifts in the resonance frequency and changes in the amplitude of the sensing signal can be improved and the electrical conductivity in the medium can be accurately determined in real time.

The first channel interface circuit 420 may be a first channel sensing terminal connected to the first electrode 212, the first sensing resonance circuit 410, or the first oscillator circuit 410a, may be a first channel sensing port including a first channel sensing terminal and also including another electric terminal connected to a common ground wire from the first sensing resonance circuit 410, or may be implemented to include an analog or digital buffer circuit connected to a first channel sensing terminal and/or a first channel sensing port.

Alternatively, the first channel interface circuit 420 may be a first channel reference terminal connected to a first reference resonance circuit (RLC) or a first reference oscillator circuit, may be a first channel reference port including a first channel reference terminal and also including another electric terminal connected to a common ground wire from a first reference resonance circuit, or may be implemented to include an analog or digital buffer circuit connected to a first channel reference element and/or a first channel reference port.

The second channel interface circuit 422 is implemented for the second channel, and has a specific configuration similar to that of the first channel interface circuit 420. A redundant description thereof will be omitted.

Although not shown in FIGS. 6 and 7, the reference resonance circuits of the two channels are not exposed to the outside, so that the electrical characteristics are not influenced regardless of changes in the ion concentration and electrical conductivity of the medium. Accordingly, the second electric signal may maintain initialized reference resonance frequencies ω1_ref and ω2_ref for each channel regardless of changes in the electrical characteristics of the medium.

The first sensing resonance circuit 410 and the second sensing resonance circuit 412 shown in FIGS. 6 and 7, respectively, may be considered to represent respective equivalent circuits. In this case, it is not necessary to include lumped RLC elements. For example, capacitance, inductance, and resistance may be independent elements, or may represent parasitic components. Furthermore, even when the first sensing resonance circuit 410 and the second sensing resonance circuit 412 are implemented using independent elements, the arrangements of the elements does not necessarily have to follow those of FIG. 6 and/or FIG. 7, and it is sufficient if they can equivalently correspond to the circuits of FIG. 6 and/or FIG. 7. Furthermore, although it is recommended that the first sensing resonance circuit 410 has the same electrical characteristics as the first reference resonance circuit, the difference in resonance frequency measured in the absence of user input may be compensated for using an offset even when there is a difference. Such processing may be performed on the second sensing resonance circuit 412 and the first reference resonance circuit in the same manner.

FIG. 8 shows the configuration of the output circuit 430 of the electrical conductivity sensing interface circuit 210 according to an embodiment of the present invention, which illustrates the mutual operations among the first sensing resonance circuit 410, a first reference resonance circuit 810, and the output circuit 430. Although FIG. 8 is shown for the operation of the first channel, those skilled in the art can infer the operation of the second channel by referring to FIG. 8. Furthermore, although FIG. 8 is shown for the output circuit 430 of FIG. 4, those skilled in the art can infer the operation of the output circuit 530 of FIG. 5 in the same manner.

The output circuit 430 of the electrical conductivity sensing interface circuit 310 according to an embodiment of the present invention includes a signal processing circuit 432 and a converter circuit 438.

The signal processing circuit 432 may generate a first channel differential signal having a first channel differential resonance frequency corresponding to the difference between a first sensing resonance frequency ω1 and a first reference resonance frequency ω1_ref through the process of processing a first channel sensing signal and a first channel reference signal. The signal processing circuit 432 may include an operator circuit 434 and a low pass filter circuit 436.

The converter circuit 438 may be connected to the output terminal of the signal processing circuit 432, and may generate a first channel output signal Ch1_out whose magnitude is proportional to the first channel differential resonance frequency.

The output circuit 430 quantitatively detects a shift in the first sensing resonance frequency ω1 of the first channel sensing signal formed in the first sensing resonance circuit 410 based on a capacitance Cx formed according to the concentration of ions contained in a medium surrounding the outside of the first electrode 212 and a parasitic resistance component Rx formed according to the electrical conductivity of the medium. In this case, when the first reference resonance frequency ω1_ref at which the first channel sensing signal has been initialized is selected to be sufficiently large, the design may be made such that the shift of the first sensing resonance frequency ω1 is mainly influenced by the capacitance Cx. Conversely, when the first reference resonance frequency ω1_ref is selected to be sufficiently small, the design may be made such that the shift of the first sensing resonance frequency ω1 is mainly influenced by the parasitic resistance component Rx.

In this case, the output circuit 430 may measure an overall impedance change attributable to the electrical conductivity and ion concentration of the medium surrounding the outside of the first electrode 212 based on the parasitic resistance component Rx and the capacitance Cx.

The reference resonance circuits of the two channels are not exposed to the outside, and thus they are not influenced by the electrical characteristics of the medium. Accordingly, the first reference resonance circuit 810 may maintain the initialized first reference resonance frequency ω1_ref regardless of changes in the electrical characteristics of the medium.

When the difference between the first sensing resonance frequency ω1 and the first reference resonance frequency ω1_ref is detected, it may be determined whether the first sensing resonance frequency ω1 of the first sensing resonance circuit 410 has shifted from the first reference resonance frequency ω1_ref, and the degree of shift may be quantitatively analyzed when the first sensing resonance frequency ω1 has shifted.

For the first channel, the difference between the first sensing resonance frequency ω1 and the first reference resonance frequency ω1_ref is detected. Although not shown in FIG. 8, the difference between the second sensing resonance frequency ω2 and the second reference resonance frequency ω2_ref may be detected for the second channel.

In this case, the sensing value of the first channel detected is ω1-ω1_ref, and the sensing value of the second channel is ω2-ω2_ref.

In this case, even when the sensing value (a shift in the resonance frequency) of each channel is not 0 but is excessively small, a first threshold that is not considered a meaningful measurement may be set in advance. When the sensing value of each channel is equal to or larger than the first threshold, the electrical characteristic, such as the electrical conductivity, of the medium may be determined to have a valid meaning different from that of an initialized state. In this case, whether the electrical conductivity has changed sufficiently to determine whether a domestic animal is estrous may be considered for a criterion for setting the first threshold.

The first sensing resonance circuit 410 and the first reference resonance circuit 810 need to be initialized to have the same impedance. In reality, there may be a minute frequency difference between the initial value of the first sensing resonance frequency ω1 and the first reference resonance frequency ω1_ref due to process conditions or various surrounding environmental variables.

This minute frequency difference value may be set as an offset. The first threshold described above may be set by considering the offset.

Furthermore, a calibration process may be performed in the output circuit 430. The calibration process may be a process in which the first sensing resonance circuit 410 and/or the first reference resonance circuit 810 are adjusted so that the offset becomes zero.

After the calibration, the first sensing resonance circuit 410 and/or the first reference resonance circuit 810 may be adjusted using means such as a means for adjusting the value of a variable resistor R′.

In another embodiment, the calibration process may be a process of detecting an offset. In this case, an offset according to each channel and situation may be stored in a separate memory or storage and processed as offset information in a future sensing process of the output circuit 430 and/or determination circuit 340.

In contrast with the present invention, the prior art employs a method of detecting a change in amplitude by applying a low-frequency alternating current signal and calculating electrical conductivity from the change in amplitude. However, this method has the problem of not being able to prevent or calibrate the contamination of measured values attributable to changes in electrical characteristics, other than electrical conductivity, according to the environment in which a sensor is located, the internal state of mucus, and/or the like.

A method of calculating electrical conductivity in multiple frequency bands may also be considered. In a prior art, there is employed a method of sequentially inputting a plurality of frequency signals through variable frequency scanning and then measuring changes in impedance. This method has the prerequisite of accurately detecting and comparing the magnitudes of signals. Accordingly, there are problems in that it takes time to measure signals and power consumption is high. A reduction in power consumption is a considerably important task for the estrous sensor that needs to be implanted into the body of a domestic animal and operate with the help of a battery for a long time.

The present invention may measure signals in multiple frequency channels without scanning for changes in frequency. With a single measurement, changes in signal amplitude and changes in resonance frequency may be measured simultaneously. Accordingly, the present invention has an advantage in that it may significantly reduce power consumption compared to the prior art. Furthermore, the present invention may obtain multiple parameters through a single scan, so that the present invention has advantages in that measured electrical conductivity values can be cross-verified and cases where there is an error or other influences in the electrical conductivity measurement can be rapidly detected.

The operator circuit 434 may generate a differential resonance frequency component signal having a frequency corresponding to the differential resonance frequency ω1-ω1_ref, which is the difference between the first sensing resonance frequency ω1 and the first reference resonance frequency ω1_ref, through the signal processing of the first channel sensing signal having the first sensing resonance frequency ω1 and the first channel reference signal having the first reference resonance frequency ω1_ref. In this case, the calculator 434 does not need to directly obtain the first sensing resonance frequency ω1 or the first reference resonance frequency ω1_ref. The frequency of the differential resonance frequency component signal obtained by the operator 434 corresponds to the differential resonance frequency ω1-ω1_ref and is smaller than each of the first sensing resonance frequency ω1 and the first reference resonance frequency ω1_ref, so that a differential high-performance signal processing circuit is not required to process the resonant frequency component signal. The signal processing of the first channel sensing signal and the first channel reference signal that generates the differential resonance frequency component signal may be performed in an analog domain, a digital domain, or an analog-digital mixed domain.

Information about whether the differential resonance frequency ω1-ω1_ref is positive or negative or which of the first sensing resonance frequency ω1 and the first reference resonance frequency ω1_ref is higher may be obtained through additional information obtained from the signal processing of the first channel sensing signal and the first channel reference signal that generates the differential resonant frequency component signal, monitoring the differential resonant frequency component signal over time, and/or the like. It may be additionally verified whether the information about which of the first sensing resonance frequency ω1 and the first reference resonance frequency ω1_ref is higher is accurate through the monitoring of the differential resonance frequency component signal over time.

The first channel output signal Ch1_out generated by the converter circuit 438 may have a voltage, a current, an amplitude, or a phase proportional to quantitative information corresponding to the difference between the first sensing resonance frequency ω1 and the first reference resonance frequency ω1_ref.

The converter circuit 438 according to one of the embodiments of the present invention may be a time-to-digital converter that generates a digitized value proportional to the frequency of a differential resonance frequency component signal. According to another embodiment of the present invention, the converter circuit 438 may be an analog voltage generator that generates an analog signal proportional to a measured frequency difference. According to another embodiment of the present invention, the converter circuit 438 may be an analog current generator that is proportional to a measured frequency difference. When the output signal is an analog voltage or an analog current, the basic offset of the analog voltage or the analog current is given when a difference in resonance frequency is 0, and the magnitude of the output signal selectively increases and decreases in proportion to a change in the difference in resonance frequency.

A frequency-to-voltage converter circuit may be used as a means of converting a difference in resonance frequency into voltage and outputting it as an analog signal. In this case, a filter capacitor may be added to remove noise. When the capacitance of the filter capacitor needs to be large, an externally exposed capacitor connection pin may be provided such that a capacitor can be connected to the outside of the electrical conductivity sensing interface circuit 310.

In some embodiments, the output circuit 430 may include a sampler and a comparator for the differential resonant frequency component signal. In this case, for the smooth operation of the output circuit 430, the sampler and the comparator may be designed to have an operating frequency sufficiently higher than the first measurement threshold for determining whether a measurement is meaningful as described above and sufficiently higher than the operating range of resonance frequency components corresponding to detection target displacements.

In embodiments of the present invention, the output circuit 430 may detect the resonance frequency information of the channel independently of the amplitude of the electrical signal of the channel (without detecting the amplitude). In this case, according to another embodiment of the present invention, a conational technology for detecting the amplitude independently of the resonance frequency is applied in parallel, and two types of sensing information obtained independently of each other (first sensing information based on the detection of the amplitude, and first reference detection information based on the detection of the resonance frequency that is independent of the amplitude) may be mutually cross-verified.

In other words, the accuracy of measurement may be further improved by comprehensively referring to the first estimation information used to estimate the state of the medium through the detection of a difference in resonance frequency and second estimation information used to estimate the state of the medium through the detection of the amplitude of a resonance signal. It may be possible to generate the state information of the medium in which the first estimation information is compensated with the second estimation information, and vice versa.

The estrous sensor 200 of the present invention may provide measurement results robust to a penetration location within the medium and the state of the medium by using the sensing electrodes 212 and 214, may detect frequency characteristics at one time without the need to scan multiple frequencies, and may detect a shift in resonance frequency and change in amplitude with a single measurement, thereby reducing measurement costs, shortening time, and improving measurement accuracy. Furthermore, the electrical conductivity of the medium may be calculated using the sensing values of the two channels.

Although not shown in FIG. 8, the signal processing circuit 432 may generate a second channel differential signal having a second channel differential resonance frequency corresponding to the difference between a second sensing resonance frequency ω2 and a second reference resonance frequency ω2_ref through the process of processing a second channel sensing signal and a second channel reference signal.

In this case, the converter circuit 438 may generate a second channel output signal Ch2_out whose magnitude is proportional to the second channel differential resonance frequency.

In the first embodiment of the present invention for the operations of the first channel and the second channel, in order to measure the state of the medium that changes in real time (to maintain a real-time property), the signal processing circuit 432 may include an operator 434 allocated to the first channel and an operator separately allocated to the second channel.

In the second embodiment of the present invention for the operations of the first channel and the second channel, a single operator 434 may perform the operations of the first channel and the second channel in a time-division manner with a focus on small size rather than real-time response.

In an embodiment of the present invention, the influence resulting from the electrical conductivity of the medium may be calculated through the minute difference between the sensing values of the two channels. In contrast with the present invention, the prior art directly measures the electrical conductivity of a medium. When the ion concentration of mucus in the body of a domestic animal is significantly out of an initially assumed range, there are many cases where it is impossible to measure electrical conductivity. The present invention may measure the overall electrical conductivity of a medium using two channel sensing values measured in an available frequency band even in a medium state to which the prior art electrical conductivity measurement is not applicable. Accordingly, the range of frequency bands and the range of medium environments that can be covered by the present invention are considerably wide.

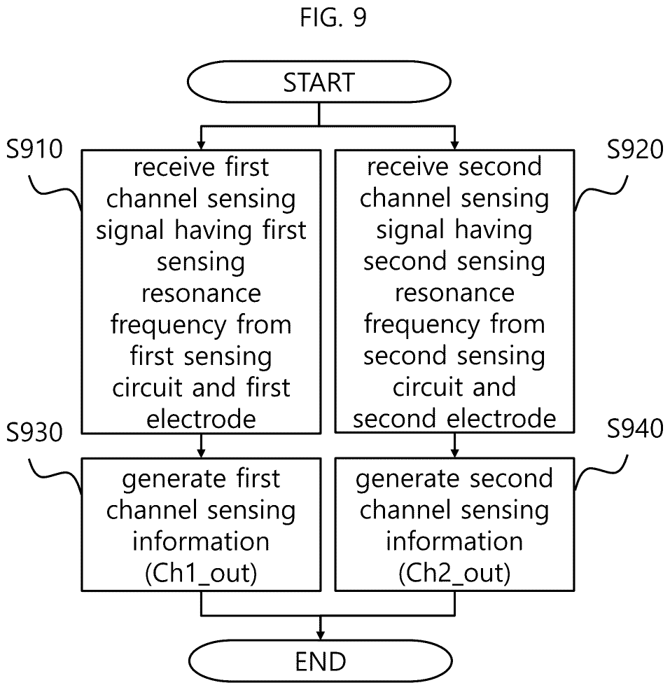

FIG. 9 is an operational flowchart illustrating an embodiment of a method of operating the electrical conductivity sensing interface circuit of FIGS. 2 and 4.

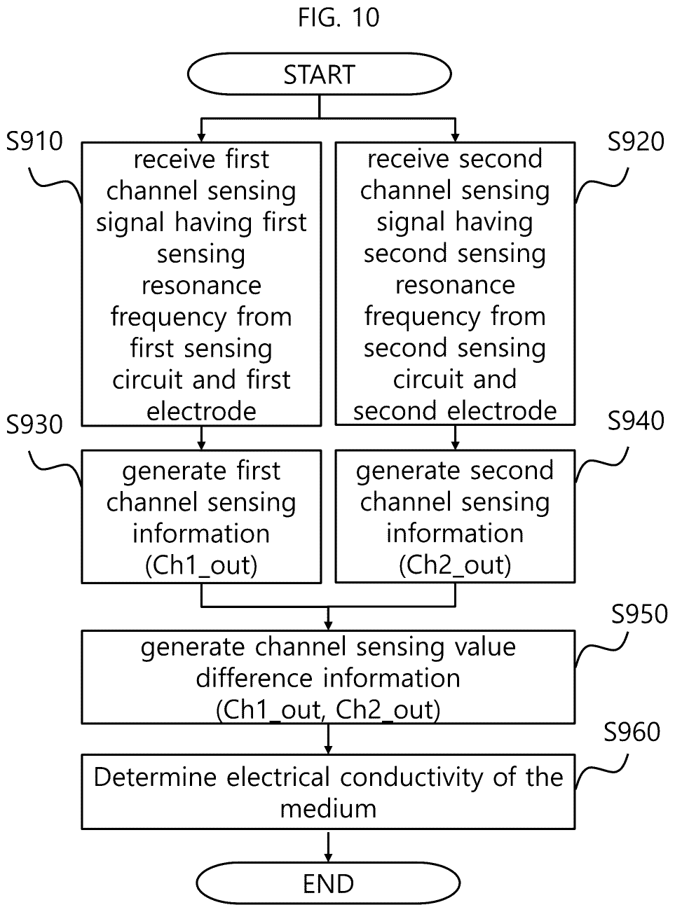

FIG. 10 is an operational flowchart illustrating an embodiment of a method of operating the estrous sensor of FIG. 2.

Referring to FIGS. 9 and 10, the electrical conductivity sensing interface circuit 310 and/or the estrous sensor 200 may receive a first channel sensing signal having a first sensing resonance frequency ω1 from the first sensing resonance circuit 410 connected to the first electrode 212 in contact with a medium (body fluid/mucus in the body of a domestic animal) in step S910.

The electrical conductivity sensing interface circuit 310 and/or the estrous sensor 200 may receive a second channel sensing signal having a second sensing resonance frequency ω2 from the second sensing resonance circuit 412 connected to the second electrode 214 in contact with the medium in step S920.

The electrical conductivity sensing interface circuit 310 and/or the estrous sensor 200 may generate first channel sensing information based on the first sensing resonance frequency ω1 using the first channel sensing signal in step S930.

The electrical conductivity sensing interface circuit 310 and/or the estrous sensor 200 may generate second channel sensing information based on the second sensing resonance frequency ω2 using the second channel sensing signal in step S940.

The first channel output signal Ch1_out, whose magnitude may be proportional to the first channel sensing information, and the second channel output signal Ch2_out, whose magnitude may be proportional to the second channel sensing information, may be transferred to the determination circuit 340 by the electrical conductivity sensing interface circuit 310.

The determination circuit 340 of the estrous sensor 200 may acquire the first channel sensing information based on the first channel output signal Ch1_out, and may acquire the second channel sensing information based on the second channel output signal Ch2_out.

The determination circuit 340 of the estrous sensor 200 may generate channel sensing value difference information Ch1_out−Ch2_out, which is the difference between the first channel sensing information and the second channel sensing information, in step S950.

The determination circuit 340 of the estrous sensor 200 may determine the electrical conductivity of the medium based on the ratio between channel interval information and the channel sensing value difference information in step S960. The channel interval information may be the difference between the first reference resonance frequency ω1_ref at which the first channel sensing signal has been initialized and the second reference resonance frequency ω2_ref at which the second channel sensing signal has been initialized.

In the embodiments of FIGS. 2 to 9, the known temperature sensors 250 and 350 may be added and used in various manners. One of the widely known temperature measurement methods is to use a negative temperature coefficient (NTC) material. Values such as a resistance value will vary depending on temperature, so that the temperature can be calculated by detecting changes in electrical signals such as voltage and current using the above phenomenon.

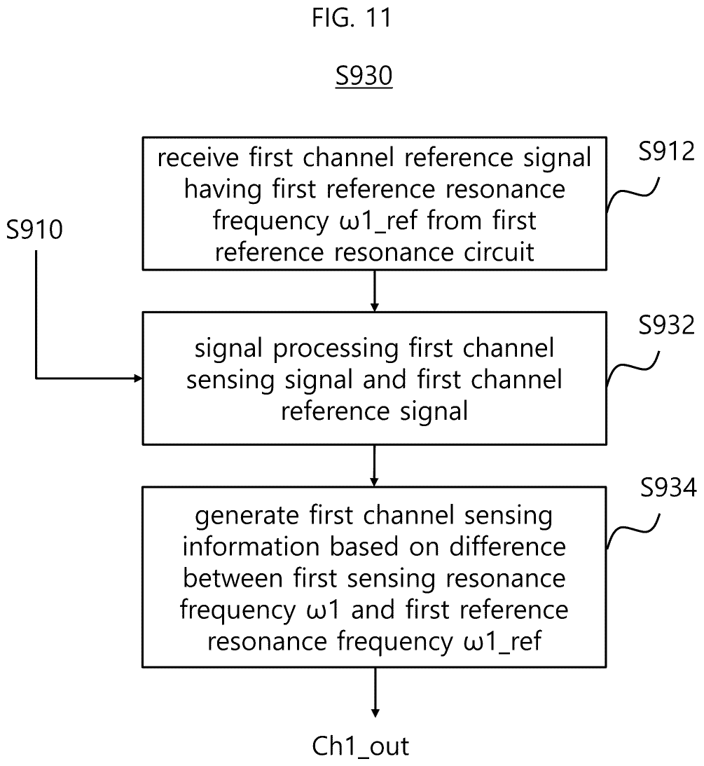

FIG. 11 is an operational flowchart illustrating step S930 of FIGS. 9 and 10 in detail.

The output circuit 430 in the electrical conductivity sensing interface circuit 210 may receive a first channel reference signal having a first reference resonance frequency ω1_ref from the first reference resonance circuit 810 in step S912.

The output circuit 430 may perform the process of signal processing the first channel sensing signal (received in step S910) and the first channel reference signal in step S932.

The output circuit 430 may generate first channel sensing information based on the difference between the first sensing resonance frequency ω1 and the first reference resonance frequency ω1_ref in step S934.

The output circuit 430 may generate and output a first channel output signal Ch1_out that may be proportional to the first channel sensing information.

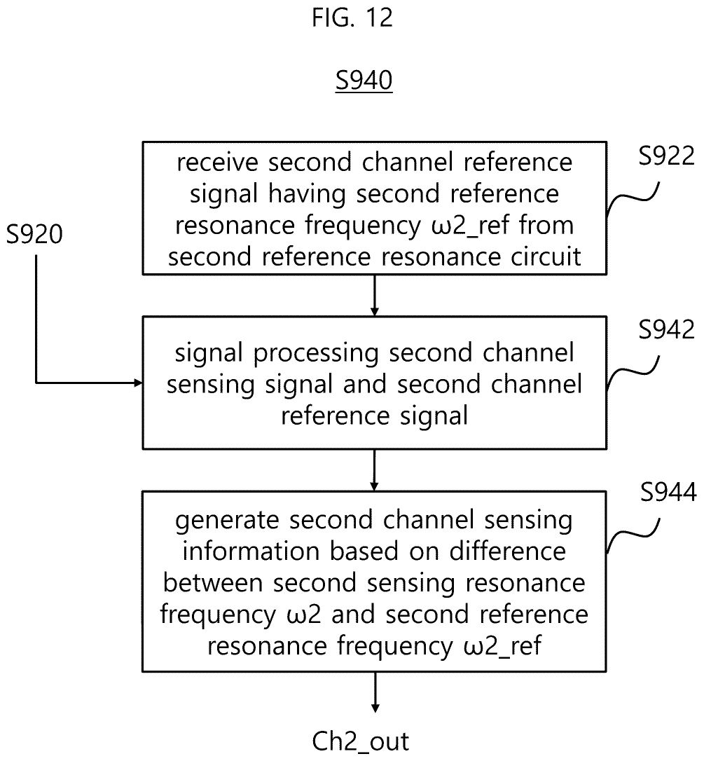

FIG. 12 is an operational flowchart illustrating step S940 of FIGS. 9 and 10 in detail.

The output circuit 430 may receive a second channel reference signal having a second reference resonance frequency ω2_ref from the second reference resonance circuit in step S922.

The output circuit 430 may perform the process of signal processing the second channel sensing signal (received in step S920) and the second channel reference signal in step S942.

The output circuit 430 may generate second channel sensing information based on the difference between the second sensing resonance frequency ω2 and the second reference resonance frequency ω2_ref in step S944.

The output circuit 430 may generate and output a second channel output signal Ch2_out that may be proportional to the second channel sensing information.

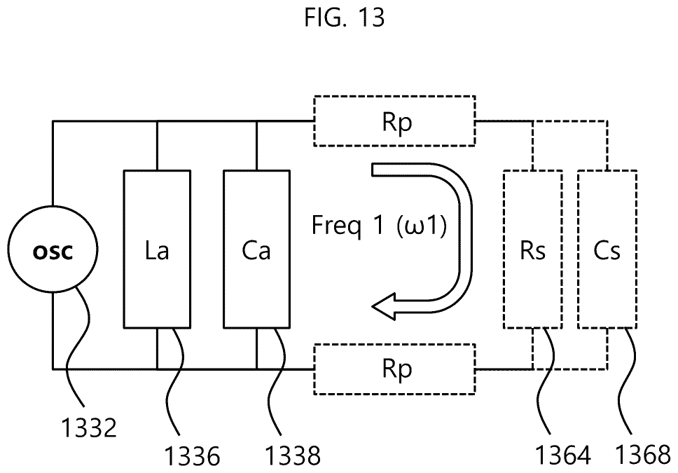

FIG. 13 is a diagram of an equivalent circuit used to illustrate the operating principle of the estrous sensor 200 according to an embodiment of the present invention. Although FIG. 13 illustrates a first channel as an example, a second channel may also be understood in a similar manner.

A first oscillator circuit 1332 may apply a first alternating current signal to a first inductor 1336 and a first capacitor 1338 constituting each of the first sensing resonance circuits 410 and 510. A parasitic resistance formed by a pair of electrode wires connecting among each of the first sensing resonance circuits 410 and 510, the first electrode 212, and the common ground is denoted by Rp in FIG. 13. A parasitic capacitance Cs 1368 and parasitic resistance Rs manifested on the first electrode 212 due to the interaction between a medium and the probe electrodes 220, 230, and 240 are combined with the impedance of the parasitic resistance Rp, inductor 1336, and first capacitor 1338 of a wire, so that the composite impedance of the estrous sensor 200 of FIG. 13 is formed. Based on this composite impedance, the first sensing resonance frequency ω1 of the first channel sensing signal formed in the equivalent circuit of FIG. 13 is determined.

In general, the parasitic capacitance Cs 1368 has a strong influence on changes in impedance in a high-frequency signal environment, is highly related to reactance (inductance, and capacitance), and is known to be influenced by the moisture content or ion concentration of the medium. It is known that a parasitic resistance Rs 1364 has a strong influence on changes in impedance in a relatively low-frequency environment and is influenced by the electrical conductivity of the medium.

In an embodiment of the present invention, the output circuit 430 and the determination circuit 340 may measure a change in the signal using frequency components in a hundreds of MHz band in the first channel, and may measure the electrical conductivity of the medium using frequency components in a few MHz or hundreds of kHz band in the second channel.

In an embodiment of the present invention, the determination circuit 130 may quantitatively detect a change in the amplitude of the channel sensing signal and a shift in the sensing resonance frequency using frequency components in a hundreds of MHz band in one channel, and may quantitatively detect a change in the amplitude of the channel sensing signal and a shift in the sensing resonance frequency using frequency components in a few MHz or hundreds of kHz band in another channel. In this case, the electrical conductivity of the medium may be measured using the difference between channel sensing values resulting from the electrical conductivity of the medium included in the information obtained from the two channels.

FIG. 14 is a diagram illustrating the operating principle by which the estrous sensor 200 according to an embodiment of the present invention determines electrical conductivity. FIG. 14 shows a method for determining electrical conductivity by using two or more channels.

In FIG. 14, for convenience of description, there is shown a graph in which the frequency domain is mapped to the X axis and the output values of channel output signals or channel sensing values are mapped to the Y axis.

The Y axis may represent the magnitudes of the voltages or currents of digital or analog signals in the case of the output values of channel output signals, or may represent amplitudes in the case of alternating current signals.

When the Y axis represents channel sensing values, they may be shift values in the sensing resonance frequency obtained in respective channels. This mapping is possible because channel output signals are signals converted to have magnitudes proportional to channel sensing values, i.e., shift values in the sensing resonance frequency.

The first reference resonance frequency ω1_ref of the first channel and the value of the first channel output signal Ch1_out are mapped as the first channel sensing information 1410 of FIG. 14.

The second reference resonance frequency ω2_ref of the second channel and the value of the second channel output signal Ch2_out are mapped as the second channel sensing information 1420 of FIG. 14.

Channel spacing information 1440, which is the difference value between the first reference resonance frequency ω1_ref and the second reference resonance frequency ω2_ref, is shown as the interval between the X coordinates of the first channel sensing information 1410 and the second channel sensing information 1420 in the graph of FIG. 14.

The difference between the first channel sensing information and the second channel sensing information may be obtained from the magnitudes of the first channel output signal Ch1_out and the second channel output signal Ch2_out. Channel sensing value difference information 1450 obtained in this manner is shown as the interval between the Y coordinates of the first channel sensing information 1410 and the second channel sensing information 1420 in the graph of FIG. 14.

The electrical conductivity of a medium may be determined based on the ratio between the channel interval information 1440 and the channel sensing value difference information 1450. This ratio is the same as the slope 1460 of a straight line 1430 passing through the first channel sensing information 1410 and the second channel sensing information 1420 in the graph of FIG. 14.

In the first embodiment in which the electrical conductivity of the medium is obtained, the value of the slope 1460 may be compared with the reference slopes of inter-channel sensing information between values measured by a precision sensor that serves as a reference, and the reference electrical conductivity of the reference sensor mapped to the sensed slope 1460 may be determined to be the sensed electrical conductivity of the medium.

In this case, the reference sensor is a precision sensor, and may store the resonance frequency shift values measured while varying the frequency under conditions including various moisture contents, ion concentrations, and electrical conductivities of the medium in a table. Meanwhile, the estrous sensor 200 of the present invention may provide the measurement condition of the reference sensor, most appropriately mapped to the sensed slope 1460, as the electrical conductivity of the medium obtained by the sensed slope 1460.

This process may be performed through comparison with a table in a rule-based manner, or may be performed by a machine learning engine that has been trained on the function of receiving a slope as input and inferring electrical conductivity through machine learning. The machine learning engine may be an artificial neural network.

In another embodiment in which electrical conductivity is obtained using two channels, the Y coordinate (the Y intercept) of the point where the straight line 1430 extends and meets the Y axis in the graph of FIG. 14 may be obtained, and a channel measurement value may be estimated on the assumption that the reference resonance frequency is 0.

In this case, electrical conductivity may be obtained by estimating the resistance component of the impedance from the obtained Y-intercept value and then inverting the resistance component.

When an alternating current signal is applied, the strengths of electric and magnetic fields continue to change over time, so that a dielectric constant needs to be treated as a complex dielectric constant and the complex dielectric constant includes a real part and an imaginary part.

In this case, the complex dielectric constant ε_comp is a function of the frequency ω, and may be represented as follows:

ε_comp(ω)=εreal(ω)−iε_imag(ω) (1)

where ε_comp (ω) is the complex dielectric constant, ε_real is a real part, ε_imag is an imaginary part, and i is an imaginary unit.

In the case where the complex dielectric constant is dealt with, when the frequency ω is maintained only in a narrow band, the dielectric constant independent of frequency, or may be approximated by a simple model function. In a general prior art, electrical conductivity is measured only at a specific frequency and another electrical characteristic, such as reactance (capacitance or inductance), is measured at a separate specific frequency.

In the present invention, the reference resonance frequencies of the two channels are set to have a significant difference. As a result, both the influences of the real and imaginary parts of a complex dielectric constant may be reflected in a measured value. In other words, when the channel interval information 1440 shown in FIG. 14 is sufficiently wide, two sensing measurement values may be designed to have a significant difference, and both the real and imaginary parts of the complex permittivity may be obtained using this difference.

This change in the complex dielectric constant is closely related to a change in the electrical characteristic attributable to a change in ion concentration in the medium (in the application of the present invention, body fluid in the body of a domestic animal, e.g., mucus in the vagina of a cow). Accordingly, when changes in the complex dielectric constant are specified in different frequency bands, the change in electrical characteristic attributable to the change in ion concentration in the medium may be specified.

According to the present invention, there may be provided the electrical conductivity sensor that may measure electrical conductivity regardless of the measurement conditions of a medium (body fluid/mucus in the body of a domestic animal)/an environment surrounding the sensor (i.e., an electrical conductivity sensor that has a large dynamic range), and there may also be provided the sensor and method for determining the estrus of a domestic animal using the electrical conductivity sensor.

According to the present invention, there may be implemented the estrous sensor and method that may stably measure the temperature and electrical conductivity of a medium regardless of the current situation of the medium, thus being significantly robust even to temporary environmental changes.

According to the present invention, there may be provided the estrous sensor that is non-destructive, may be used semi-permanently, provides device stability, and does not require a configuration for varying the frequency of an input electrical signal, thereby significantly reducing manufacturing costs compared to conventional estrous sensors.

According to the present invention, there may be implemented the estrous sensor that has a circuit and operation method capable of effectively detecting a shift in resonance frequency. Furthermore, according to the present invention, there may be implemented the medium monitoring sensor that does not require the process of varying the frequency of an input electrical signal, thereby shortening the time required to sense electrical conductivity in a medium.

According to the present invention, there may be provided the estrous sensor that is designed to use one or two sensing electrodes depending on the embodiment and is also designed such that, when two electrodes are used, both the sensing electrodes participate in the process of measuring the electrical conductivity of a medium. Accordingly, According to the present invention, there may be provided the estrous sensor that does not limit the measurement conditions/range of a medium under which characteristic parameters, such as moisture content and salts, of the medium can be monitored, may deal with a medium in various environments, and may increase the correlation between measured values over time.

Although omitted in the drawings in connection with the embodiments of FIGS. 2 to 13, a processor and memory are electronically connected to the individual components, and the operations of the individual components may be controlled or managed by the processor.

The processor may mean a central processing unit (CPU), a graphics processing unit (GPU), or a dedicated processor on which the methods according to exemplary embodiments of the present disclosure are performed.

Each of the memory and the storage device may include at least one of a volatile storage medium and a non-volatile storage medium. For example, the memory may include at least one of a read only memory (ROM) and a random access memory (RAM).

In addition, the computing system may include the communication interface that performs communication through a wireless network.

In addition, the respective components in the computing system may be connected by the bus to communicate with each other.

For example, the computing system including the processor of the present disclosure may be a desktop computer, a laptop computer, a notebook, a smart phone, a tablet PC, a mobile phone, a smart watch, a smart glass, e-book reader, a portable multimedia player (PMP), a portable gaming device, a navigation device, a digital camera, a digital multimedia broadcasting (DMB) player, a digital audio recorder, a digital audio player, a digital video recorder, a digital video player, a personal digital assistant (PDA), and the like having communication capability.

The operations of the method according to the exemplary embodiment of the present disclosure can be implemented as a computer readable program or code in a computer readable recording medium. The computer readable recording medium may include all kinds of recording apparatus for storing data which can be read by a computer system. Furthermore, the computer readable recording medium may store and execute programs or codes which can be distributed in computer systems connected through a network and read through computers in a distributed manner.

The computer readable recording medium may include a hardware apparatus which is specifically configured to store and execute a program command, such as a ROM, RAM or flash memory. The program command may include not only machine language codes created by a compiler, but also high-level language codes which can be executed by a computer using an interpreter.

Although some aspects of the present disclosure have been described in the context of the apparatus, the aspects may indicate the corresponding descriptions according to the method, and the blocks or apparatus may correspond to the steps of the method or the features of the steps. Similarly, the aspects described in the context of the method may be expressed as the features of the corresponding blocks or items or the corresponding apparatus. Some or all of the steps of the method may be executed by (or using) a hardware apparatus such as a microprocessor, a programmable computer or an electronic circuit. In some embodiments, one or more of the most important steps of the method may be executed by such an apparatus.

In some exemplary embodiments, a programmable logic device such as a field-programmable gate array may be used to perform some or all of functions of the methods described herein. In some exemplary embodiments, the field-programmable gate array may be operated with a microprocessor to perform one of the methods described herein. In general, the methods are preferably performed by a certain hardware device.

The description of the disclosure is merely exemplary in nature and, thus, variations that do not depart from the substance of the disclosure are intended to be within the scope of the disclosure. Such variations are not to be regarded as a departure from the spirit and scope of the disclosure. Thus, it will be understood by those of ordinary skill in the art that various changes in form and details may be made without departing from the spirit and scope as defined by the following claims.

What is claimed is:

1. An estrous sensor that is implanted into a body of a domestic animal and detects estrus of the domestic animal, the estrous sensor comprising:
a first channel interface circuit configured to receive a first channel sensing signal having a first sensing resonance frequency from a first sensing resonance circuit connected to a first electrode in contact with body fluid of the domestic animal within the body of the domestic animal;
an output circuit configured to:
receive the first channel sensing signal via the first channel interface circuit; and
generate a first channel output signal;
a temperature measurement circuit configured to detect temperature within the body of the domestic animal; and
a determination circuit configured to:
receive the first channel output signal;
receive the temperature within the body of the domestic animal from the temperature measurement circuit; and
determine whether the domestic animal is estrous,
wherein the output circuit is further configured to:
generate a first channel sensing intermediate signal having a first channel differential sensing frequency, which is a difference between a first reference resonance frequency at which the first channel sensing signal has been initialized and the first sensing resonance frequency, by using a process of processing the first channel sensing signal;
generate first channel sensing information based on the first channel differential sensing frequency; and
output the first channel output signal having a magnitude corresponding to electrical conductivity of the body fluid of the domestic animal based on the first channel sensing information, and
wherein the determination circuit is further configured to:
detect the electrical conductivity of the body fluid of the domestic animal by the first channel output signal; and
determine whether the domestic animal is estrous based on the electrical conductivity of the body fluid of the domestic animal and the temperature within the body of the domestic animal.

2. The estrous sensor of claim 1, further comprising a second channel interface circuit configured to receive a second channel sensing signal having a second sensing resonance frequency from a second sensing resonance circuit connected to a second electrode in contact with the body fluid of the domestic animal within the body of the domestic animal,
wherein the output circuit is further configured to:
receive the second channel sensing signal via the second channel interface circuit, and generate a second channel output signal;
generate a second channel sensing intermediate signal having a second channel differential sensing frequency, which is a difference between a second reference resonance frequency, at which the second channel sensing signal has been initialized, and the second sensing resonance frequency using a process of processing the second channel sensing signal;
generate second channel sensing information based on the second channel differential sensing frequency; and
output the second channel output signal based on the second channel sensing information, and
wherein the determination circuit is further configured to:
receive the second channel output signal;
generate channel interval information, which is a difference between the first reference resonance frequency and the second reference resonance frequency;
generate channel sensing value difference information, which is a difference between the first channel sensing information and the second channel sensing information; and
detect the electrical conductivity of the body fluid of the domestic animal based on a ratio between the channel sensing value difference information and the channel interval information, and determine whether the domestic animal is estrous based on the electrical conductivity of the body fluid of the domestic animal and the temperature within the body of the domestic animal.

3. The estrous sensor of claim 1, wherein the output circuit is further configured to:
generate first channel sensing additional information corresponding to an amplitude of the first channel sensing signal; and
output a first channel additional output signal having a magnitude corresponding to the electrical conductivity of the body fluid of the domestic animal based on the first channel sensing additional information, and
wherein the determination circuit is further configured to:
cross-verify a first determination value corresponding to the electrical conductivity of the body fluid of the domestic animal detected by the first channel output signal by using a second determination value corresponding to the electrical conductivity of the body fluid of the domestic animal detected by the first channel additional output signal;
generate a third determination value of the electrical conductivity of the body fluid of the domestic animal based on results of the cross-verification; and
determine whether the domestic animal is estrous based on the third determination value and the temperature within the body of the domestic animal.

4. An estrous sensor that is implanted into a body of a domestic animal and detects estrus of the domestic animal, the estrous sensor comprising:
a first channel interface circuit configured to receive a first channel sensing signal having a first sensing resonance frequency from a first sensing resonance circuit connected to a first electrode in contact with body fluid of the domestic animal within the body of the domestic animal; and
an output circuit configured to:
receive the first channel sensing signal via the first channel interface circuit; and
generate a first channel output signal;
wherein the output circuit is further configured to:
generate a first channel sensing intermediate signal having a first channel differential sensing frequency, which is a difference between a first reference resonance frequency at which the first channel sensing signal has been initialized and the first sensing resonance frequency, by using a process of processing the first channel sensing signal;
generate first channel sensing information based on the first channel differential sensing frequency;
output the first channel output signal having a magnitude corresponding to electrical conductivity of the body fluid of the domestic animal based on the first channel sensing information; and
transfer the channel output first signal to a determination circuit configured to detect temperature within the body of the domestic animal by a temperature measurement circuit so that the determination circuit detects the electrical conductivity of the body fluid of the domestic animal by the first channel output signal and determines whether the domestic animal is estrous based on the electrical conductivity of the body fluid of the domestic animal and the temperature within the body of the domestic animal.

5. An estrus determination method using an estrous sensor that is implanted into a body of a domestic animal and detects estrus of the domestic animal, the estrus determination method comprising:
receiving a first channel sensing signal having a first sensing resonance frequency from a first sensing resonance circuit connected to a first electrode in contact with body fluid of the domestic animal within the body of the domestic animal;
detecting temperature within the body of the domestic animal;
generating a first channel sensing intermediate signal having a first channel differential sensing frequency, which is a difference between a first reference resonance frequency at which the first channel sensing signal has been initialized and the first sensing resonance frequency, by using a process of processing the first channel sensing signal;
generating first channel sensing information based on the first channel differential sensing frequency;
generating the first channel output signal having a magnitude corresponding to electrical conductivity of the body fluid of the domestic animal based on the first channel sensing information;
detecting the electrical conductivity of the body fluid of the domestic animal by the first channel output signal; and
determining whether the domestic animal is estrous based on the electrical conductivity of the body fluid of the domestic animal and the temperature within the body of the domestic animal.

6. The estrus determination method of claim 5, further comprising, before detecting the electrical conductivity of the body fluid of the domestic animal:
receiving a second channel sensing signal having a second sensing resonance frequency from a second sensing resonance circuit connected to a second electrode in contact with the body fluid of the domestic animal within the body of the domestic animal;
receiving the second channel sensing signal via the second channel interface circuit, and generating a second channel output signal;
generating a second channel sensing intermediate signal having a second channel differential sensing frequency, which is a difference between a second reference resonance frequency, at which the second channel sensing signal has been initialized, and the second sensing resonance frequency using a process of processing the second channel sensing signal;
generating second channel sensing information based on the second channel differential sensing frequency; and
generating the second channel output signal based on the second channel sensing information,
wherein detecting the electrical conductivity of the body fluid of the domestic animal comprises:
generating channel interval information, which is a difference between the first reference resonance frequency and the second reference resonance frequency;
generating channel sensing value difference information, which is a difference between the first channel sensing information and the second channel sensing information; and
detecting the electrical conductivity of the body fluid of the domestic animal based on a ratio between the channel sensing value difference information and the channel interval information.

7. The estrus determination method of claim 5, further comprising, before detecting the electrical conductivity of the body fluid of the domestic animal, generating first channel sensing additional information corresponding to an amplitude of the first channel sensing signal and outputting a first channel additional output signal having a magnitude corresponding to the electrical conductivity of the body fluid of the domestic animal based on the first channel sensing additional information,
wherein detecting the electrical conductivity of the body fluid of the domestic animal comprises:
cross-verifying a first determination value corresponding to the electrical conductivity of the body fluid of the domestic animal detected by the first channel output signal by using a second determination value corresponding to the electrical conductivity of the body fluid of the domestic animal detected by the first channel additional output signal; and
generating a third determination value of the electrical conductivity of the body fluid of the domestic animal as a final determination value of the electrical conductivity of the body fluid of the domestic animal based on results of the cross-verification.

8. An estrus determination method using an estrous sensor that is implanted into a body of a domestic animal and detects estrus of the domestic animal, the estrus determination method comprising:
receiving a first channel sensing signal having a first sensing resonance frequency from a first sensing resonance circuit connected to a first electrode in contact with body fluid of the domestic animal within the body of the domestic animal;
generating a first channel sensing intermediate signal having a first channel differential sensing frequency, which is a difference between a first reference resonance frequency at which the first channel sensing signal has been initialized and the first sensing resonance frequency, by using a process of processing the first channel sensing signal;
generating first channel sensing information based on the first channel differential sensing frequency;
outputting the first channel output signal having a magnitude corresponding to electrical conductivity of the body fluid of the domestic animal based on the first channel sensing information; and
transferring the first channel output signal to an estrous determination circuit so that the estrous determination circuit detects the electrical conductivity of the body fluid of the domestic animal by the first channel output signal and determines whether the domestic animal is estrous based on the electrical conductivity of the body fluid of the domestic animal and the temperature within the body of the domestic animal.
